SEQUENCE LISTING (4 sequences)

SEQ ID NO: 1 - protein, 168 aa, Artificial Sequence
  synthetic construct
  MISC_FEATURE (1)..(1): Xaa at position 1 is absent or Met;
  MISC_FEATURE (2)..(2): Xaa at position 2 is absent or is Ala, Cys, Asp, Glu, Phe, Gly, His, Ile, Leu, Met, Asn, Gln, Arg, Ser, Thr, Val, Trp, or Tyr
  MISC_FEATURE (26)..(26): Xaa at position 26 is Asn, Lys or Glu;
  MISC_FEATURE (40)..(40): Xaa at position 40 is Asn, Lys or Glu;
  MISC_FEATURE (78)..(78): Xaa at position 78 is Arg or Glu;
  MISC_FEATURE (85)..(85): Xaa at position 85 is Asn, Lys or Glu;
  MISC_FEATURE (90)..(90): Xaa at position 90 is Trp, Lys, Pro, or Arg;
  MISC_FEATURE (128)..(128): Xaa at position 128 is Ser, Thr, Lys or Glu;
  MISC_FEATURE (141)..(141): Xaa at position 141 is Arg or Glu;
  MISC_FEATURE (156)..(156): Xaa at position 156 is Lys or Glu; and
  MISC_FEATURE (168)..(168): Xaa at position 168 is Arg, absent, or any other amino acid.
XXAPPRLICDSRVLERYLLEAKEAEXITTGCAEHCSLNEXITVPDTKVNFYAWKRMEVGQQAVEVWQGLALLSEAVLXGQALLVXSSQPXEPLQLHVDKAVSGLRSLTTLLRALGAQKEAISPPDAAXAAPLRTITADTFXKLFRVYSNFLRGKLXLYTGEACRTGDX

SEQ ID NO: 2 - protein, 193 aa, homo sapiens
MGVHECPAWLWLLLSLLSLPLGLPVLGAPPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGDR

SEQ ID NO: 3 - DNA, 498 bp, homo sapiens
gctccaccacgtcttatttgtgattctcgtgttcttgaacgttacctgctggaagctaaagaagctgaaaacatcaccaccggttgcgctgaacactgctccctgaacgaaaacatcaccgttccggacaccaaagttaacttctacgcttggaaacgtatggaagttggtcagcaggctgttgaagtttggcagggtctggctctgctgtccgaagctgttctgcgtggtcaggctctgctggttaactcctcccagccgtgggaaccgctgcagctgcacgttgacaaagctgtttccggtctgcgttccctgaccaccctgctgcgtgctctgggtgctcagaaagaagctatctccccgccggacgctgcttccgctgctccgctgcgtaccatcaccgctgacaccttccgtaaactgttccgtgtttactccaacttcctgcgtggtaaactgaaactgtacaccggtgaagcttgccgtaccggtgactga

SEQ ID NO: 4 - protein, 165 aa, Artificial Sequence
  synthetic construct
APPRLICDSRVLERYLLEAKEAENITTGCAEHCSLNENITVPDTKVNFYAWKRMEVGQQAVEVWQGLALLSEAVLRGQALLVNSSQPWEPLQLHVDKAVSGLRSLTTLLRALGAQKEAISPPDAASAAPLRTITADTFRKLFRVYSNFLRGKLKLYTGEACRTGD

(19) United States
(12) Patent Application Publication
(10) Pub. No.: US 2007/0100133 A1
(43) Pub. Date: May 3, 2007

(54) Erythropoietic compounds

(76) Inventors: John Michael Beals, Indianapolis, IN (US); Wolfgang Glaesner, Indianapolis, IN (US); Radmila Micanovic, Indianapolis, IN (US); Rohn Lee Millican, Indianapolis, IN (US); Derrick Ryan Witcher, Fishers, IN (US)

Correspondence Address: ELI LILLY & COMPANY, PATENT DIVISION, P.O. BOX 6288, INDIANAPOLIS, IN, 46206-6288, US

(21) Appl. No.: 11/521,059

(22) Filed: Sep. 14, 2006

Related U.S. Application Data
(63) Continuation of application No. 09/856,451, filed on Jun. 25, 2002, now abandoned.
(60) Provisional application No. 60/110,289, filed on Nov. 30, 1998.

(51) Int. Cl. C07K 14/51

(52) U.S. Cl. 530/350

(57) ABSTRACT

The present invention addresses the need for better pharmaceutical agents for teating anemias by providing polymer derivatized non-glycosylated erythropoietic compounds which show stability and bioactivity in vivo. The invention further provides methods for preparing these derivatived proteins which involves the use of a linkerless aldehyde modification process.

The present invention is in the field of human medicine, particularly in the treatment of conditions treatable by stimulation of erythropoiesis, such as anemia. More specifically, the invention relates to polymer-derivatized, non-glycosylated proteins that cause an increase in blood-hematocrit when administered to a patient.

Anemia is a condition characterized by a lower than normal volume or number-of red blood cells, which are also known as erythrocytes, in the blood. One measure of the volume of red blood cells in blood is the hematocrit measurement. Blood hematocrit is the ratio, commonly expressed as a percentage, of the volume of packed cells in a sample to the total volume of the sample. Normal hematocrit levels range between 36% and 53%. Generally, a blood hematocrit below 36% is indicative of anemia.

Anemia is wide-spread in all societies, frequently associated with such conditions as renal failure, beta thalassemia, pregnancy, menstrual disorders, spinal cord injury, acute blood loss, hypoxia, aging, HIV infection associated with AZT therapy, and different neoplastic disease states accompanied by abnormal erythropoiesis, among many others.

Red blood cells are continuously being formed and destroyed in the body. Anemia arises when formation cannot keep pace with the destruction and loss of red blood cells. Therapy of anemia is directed toward either reducing destruction and loss of red blood cells or toward replacing or increasing the formation of red blood cells, or both. An object of the present invention is to increase the formation of red blood cells.

Red blood cell formation occurs through a complex process known as erythropoiesis. This process, which occurs in the bone marrow, begins when a fraction of primitive multi-potent hematopoietic stem cells becomes committed to the red blood cell lineage. The stem cells-first form burst forming units-erythroid (BFU-E), then in succession, colony-forming units-erythroid (CFU-E), normoblasts, erythroblasts, reticulocytes, and finally mature erythrocytes. Because of its complexity and essentiality, many control systems are needed to properly regulate erythropoiesis. Erythropoietin is one such control.

Erthropoietin (EPO) is a hormone that stimulates erythrbpoiesis. EPO is produced in the kidneys, is secreted into the blood stream and stimulates the differentiation of precursor cells into erythrocytes in the bone marrow. The mechanism by which EPO stimulates erythropoiesis involves the binding of EPO to specific cell-surface receptors. Activation of the EPO receptor triggers intracellular signaling events including phosphorylation of the receptor followed by activation of the JAK-STAT, RAS, and PI3 kinase pathways. These signaling pathways trigger cells to undergo proliferation and differentiation and to block apoptosis.

EPO is a glycoprotein, having a protein portion and a carbohydrate portion. The protein portion of the predominant allelic variant of human EPO consists of 166 amino acids, and the sequence is known. EPO has been produced by recombinant DNA techniques (rHuEPO). Recombinant human EPO produced in Chinese Hamster Ovary (CHO) cells and other cell lines has only 165 amino acids, lacking an arginine at position 166 [L. Owers-Narhi, et al., J. of Biol. Chem. 266:23022-23026 (1991)].

Human EPO has an apparent molecular weight of around 30.4 kDa. About 40% of the apparent molecular weight of EPO is due to carbohydrate. EPO has three N-linked oligosaccharide chains at amino acid positions 24, 38, and 83 and one O-linked oligosaccharide chain at position 126 of the mature protein. A-high degree of heterogeneity in the branching and sialic acid content has been observed both at each N-linked glycosylation site and between sites.

The role of carbohydrate is complex. Studies have shown that proper glycosylation of specific sites is critical for proper biosynthesis and secretion of EPO. It is thought to promote correct folding during protein expression, and to protect EPO and recombinant glycosylated erythropoietic proteins from degradation during their biosynthesis, secretion, and circulation.

The carbohydrate portion of the molecule also has a great influence on in vivo activity. Certain processes that remove EPO and recombinant glycosylated erythropoietic proteins from the circulation are affected by the structure of the carbohydrate attached to EPO. Various studies have shown that the removal of terminal sialic acids from EPO destroys its in vivo activity. This is due in part to the fact that desialylated EPO is cleared from the circulation much faster than fully sialylated EPO. On the other hand, the in vitro activity of EPO actually increases with desialylation. This is most likely due to an increased affinity for the receptor.

Several pharmaceutical products contain recombinant glycosylated erythropoietic proteins. Although none of these proteins have exactly the same amino acid or carbohydrate structure as does human EPO, they differ very little structurally from human EPO, and have been found to be effective therapeutically for treating certain anemias. However, these recombinant glycosylated erythropoietic proteins have not been optimized as therapeutic entities.

For example, these proteins must be administered intravenously or subcutaneously at fairly frequent intervals in order to maintain their stimulation of erythropoiesis. The recombinant molecule's natural properties limit the performance of the drug to traditional drug delivery systems. It would benefit the treatment of anemia, and reduce the discomfort and inconvenience associated with known recombinant glycosylated erythropietic proteins to provide a pharmaceutical agent that could be administered less frequently, optionally by alternate routes of administration, and that would be more stable in order to permit longer-term storage. Additional benefits of longer action would be a more natural pharmacokinetic profile, extended efficacy beyond the maximal reimbursement hematocrit level of 36, and potentially, reduction of adverse event's such as hypertension. Thus, a need exists to develop agents that stimulate erythropoiesis, are more optimal in duration of effect, and are more stable pharmaceutically.

One approach has been to alter the carbohydrate content or structure of recombinant glycosylated erythropoietic proteins, e.g., novel erythrdpoiesis stimulating protein (NESP). Adding additional sites for glycosylation might be expected to enhance the stability of EPO in vivo and extend its half-life in the circulation. Additional glycosylation, however, is not necessarily a good approach in each case because it might result in a molecule less able to, bind to the EPO receptor or more likely to be removed from the circulaation by glycoprotein, clearance mechanisms, and therefore, less suited to stimulate erythropoiesis. An additional concern is that increasing glycosylation of EPO may negatively impact bioavailability of delivered protein.

Proteins are often-administered using parenteral, pulmonary, oral, nasal, or transdermal methodologies. Consequently, the quantity of material administered needs to be altered to offset complications associated with reduced bioavailability, altered pharmacokinetics, and altered pharmacodynamics. Furthermore, the demands of exogenous administration require protein properties not necessarily intrinsic in the native protein, such as solubility that exceed in vivo concentrations by an order of magnitude or greater. Finally, the formulation demands of an exogenously-administered therapeutic protein, necessary to elicit an in vivo response, can adversely impact the toxicological effect of the macromolecule.

To circumvent these problems and address the need for better pharmaceutical agents for treating anemias, non-glycosylated erythropoietic proteins derivatized with polyethylene glycol, were invented. Surprisingly, such molecules have been found to retain the ability of EPO to increase hematocrit levels in vivo.

An article by Francis, et al., suggests that the methods of the present invention result in an EPO molecule that has low bioactivity. Intl. J. Hem. (1998) 68:1-18. The authors state that the process of the present invention, using PEG-acetaldehyde as the activated polymer, results in very poor conservation of activity when wild-type EPO is modified. Francis, et al., do suggest, however, that tresylmonomethoxy-polyethyleneglycol (TMPEG) may be used to PEGylated GM-CSF and non-glycosylated wild type human erythropoietin. Yet, no methods regarding specific conditions necessary to maintain in vivo bioactivity and at the same time promote PEGylation are provided. The authors also do not characterize any of the non-glycosylated PEGylated EPO proteins that were produced nor do they discuss any of the non-glycosylated analogs claimed in the present invention. The reference suggests only that it is possible to maintain in vitro activity from non-glycosylated PEGylated EPO. Yet, as discussed extensively below, whether a particular PEGylated protein has in vitro activity is essentially irrelevant with respect to whether that protein can function in vivo.

Contrary to the teachings by Francis, et al., however, the present invention provides polymer-derivatized non-glycosyated erythropoietic compounds that can be produced using a linkerless aldehyde modification process and that show stability and bioactivity in vivo.

In one aspect, the present invention includes non-glycosylated proteins of the Formula (I):

```
(I)
(SEQ ID NO:1)
−2 −1
Xaa Xaa
1 5 10 15
Ala Pro Pro Arg Leu Ile Cys Asp Ser Arg Val Leu Glu Arg Tyr Leu
                        20 25 30
Leu Glu Ala Lys Glu Ala Glu Xaa Ile Thr Thr Gly Cys Ala Glu His
                  35 40 45
Cys Ser Leu Asn Glu Xaa Ile Thr Val Pro Asp Thr Lys Val Asn Phe
        50 55 60
Tyr Ala Trp Lys Arg Met Glu Val Gly Gln Gln Ala Val Glu Val Trp
65 70 75 80
Gln Gly Leu Ala Leu Leu Ser Glu Ala Val Leu Xaa Gly Gln Ala Leu
                                85 90 95
Leu Val Xaa Ser Ser Gln Pro Xaa Glu Pro Leu Gln Leu His Val Asp
                        100 105 110
Lys Ala Val Ser Gly Leu Arg Ser Leu Thr Thr Leu Leu Arg Ala Leu
                115 120 125
Gly Ala Gln Lys Glu Ala Ile Ser Pro Pro Asp Ala Ala Xaa Ala Ala
        130 135 140
Pro Leu Arg Thr Ile Thr Ala Asp Thr Phe Xaa Lys Leu Phe Arg Val
145 150 155 160
Tyr Ser Asn Phe Leu Arg Gly Lys Leu Xaa Leu Tyr Thr Gly Glu Ala
                                165
Cys Arg Thr Gly Asp Xaa
```

wherein:

Xaa at position −2 is absent or Met;

Xaa at position −1 is absent or is Ala, Cys, Asp, Glu, Phe, Gly, His, Ile, Leu, Met, Asn, Gln, Arg, Ser, Thr, Val, Trp, or Tyr;

Xaa at position 24 is Asn, Lys or Glu;

Xaa at position 38 is Asn, Lys or Glu;

Xaa at position 76,is Arg or Glu;

Xaa at position 83 is Asn, Lys or Glu;

Xaa at position 88 is Trp, Lys, Pro, or Arg;

Xaa at position 126 is Ser, Thr, Lys or Glu;

Xaa at position 139 is Arg or Glu;

Xaa at position 154 is Lys or Glu; and

Xaa at position 166 is Arg, absent, or any other amino acid.

Non-glycosylated erythropoietin analogs include proteins selected from the group consisting of: a) NGE; b) NGE[5E]; c) MR-NGE; d) MR-NGE-88E; e) MR-NGE-88K; f) MR-NGE-88P; g). MR-NGE-88S; h) MR-NGE[4E]; i) MR-NGE[5E]; j) MR-NGE[5K]; k) MR-NGE[W5E); and 1) MR-NGE[W5K]. These Non-glycosylated erythropoietin analogs may have any amino acid at position 166or may have amino acid 166 deleted.

These non-glycosylated EPO analogs do not themselves cause a significant increase in hematocrit, but they acquire that property once they are derivatized suitably with polyethylene glycol polymers. They are therefore useful industrially as starting materials for preparing other compositions of the present invention.

The invention further provides isolated nucleic acids encoding the analogs of Formula I as well as vectors and host cells comprising these nucleic acids. The invention also encompasses a transgenic or chimeric non-human animal or plant, comprising host cells capable of expressing the EPO analogs of Formula I.

In another aspect, the invention provides erythropoie tic compounds having a protein portion and a polymer portion, wherein the protein portion is selected from the group consisting of: non-glycosylated human erythropoietin and non-glycosylated erythropoietin analogs and wherein the polymer portion consists of from 1 to 5 polymer chains of the formula: [R—O—(CH₂CH₂—O )ₓ—(CH₂)_{y}—NH, wherein R is H or C₁ to C₄ alkyl, X is a number from about 70 to about 1200, and Y is a number from 1 to 4; and the polymer chain is covalently bonded to the protein portion by a secondary amine bond, or a pharmaceutically-acceptable salt thereof.

The invention further provides a method for preparing polymer-derivatized, non-glycosylated erythropoietic compounds, comprising: a) adding a polyethylene glycol-aldehyde polymer to a solution containing non-glycosylated erythropoietin under conditions that permit the formation of an imine bond between an amino group of the protein and the aldehyde group of the polymer; and b) adding a reducing agent to reduce the imine bond to a secondary amine bond.

The invention also provides a method for increasing the hematocrit level in a mammal comprising administering a therapeutically effective amount of a polymer-derivatized, non-glycosylated erythropoietic compound to the mammal.

Because the modified analogs of the present invention do not have attached carbohydrate groups, they are less likely to be cleared from the circulation by natural glycosylation-mediated routes. Addition of polyethylene polymers to several different proteins has been shown to improve their pharmaceutical properties. Yet, there are very few polymer-modified proteins that have been approved as therapeutics. Addition of polyethylene glycol groups to proteins has been problematic in that the coupling/activation step can cause substantial loss of biological activity. In addition, the inability to control the coupling reaction has resulted in the addition of polymers at positions which cause steric hindrance and preclusion of protein-receptor binding.

The present invention, however, provides polyethylene glycol derivatized non-glycosylated EPO analogs which show stability and bioactivity in vivo. Because the carbohydrate chains attached to EPO have been shown to affect the protein's stability, solubility, and in vivo activity, it is surprising that stable non-glycosylated EPO analogs can be produced and modified to exhibit improved therapeutic qualities.

Moreover, the modification of the positively charged residues on non-glycosylated EPO (NGE) and NGE analogs to produce a longer-acting therapeutic is problematic and non-trivial in light of the proposed interactions between ligand and receptor. It has been proposed that EPO receptor specificity is mediated through the positively charged (basic) residues on rHuEPO interacting with the negatively charged (acidic) residues on the EPO receptor. [S. Elliott, et al., (1997) Mapping the active site of recombinant human erythropoietin, Blood 89:493-502). Consequently, the finding that modification of the positively-charged residues on EPO and EPO analogs enhances in vivo activity is counter-intuitive, surprising, and unexpected.

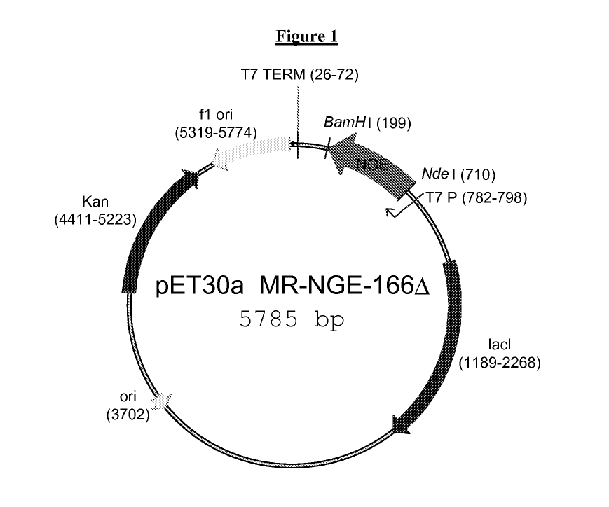

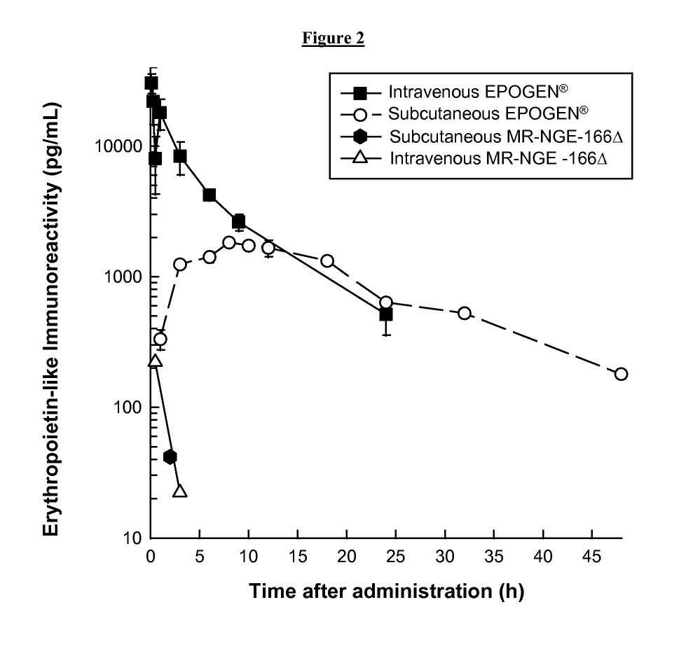

FIG. 1: Expression vector for NGE and NGEA.

FIG. 2: Blood concentrations of immunoreactive substances in mice following, a single subcutaneous or intravenous dose. (1 μg/kg) of either MR-NGE-166Δ or a commercial glycosylated EPO.

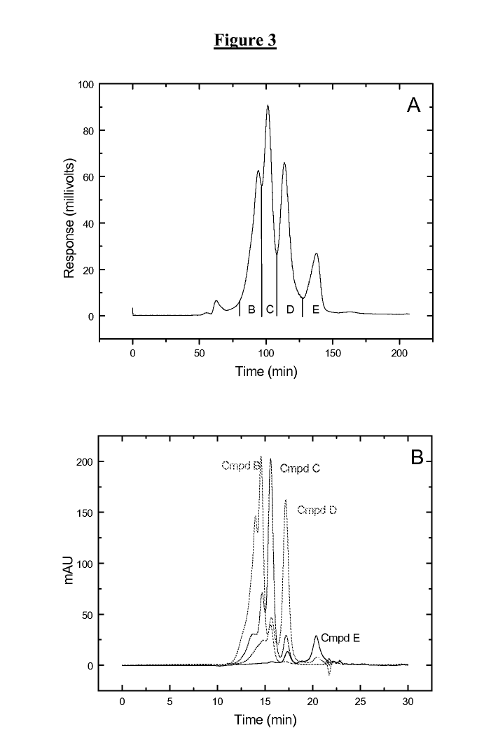

FIG. 3: (A) Preparative size exclusion chromatogram (SEC) of the reaction-mixture from a synthesis of PEG (5 kDa)-aldehyde/MR-NGE-24K, 38K, 83K, 126K, 166Δ. The pooled fractions are designated by letter. (B) Analytical HPLC size exclusion chromatogram of the pooled fractions.

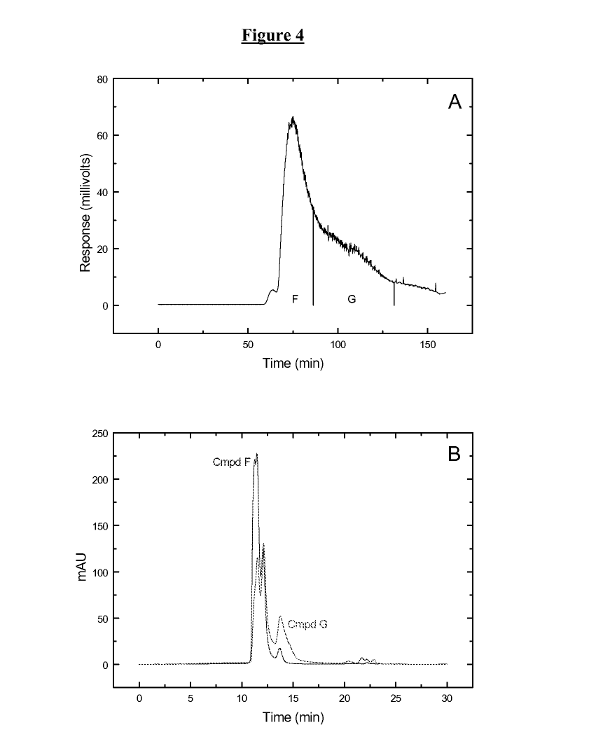

FIG. 4: Preparative size exclusion chromatogram (SEC) of the reaction mixture from a synthesis of PEG (20 kDa)-aldeyde/MR-NGE-24K, 38K, 83K, 126K, 166Δ. The pooled fractions are designated by letter. (B) Analytical HPLC size exclusion chromatogram of the pooled fractions.

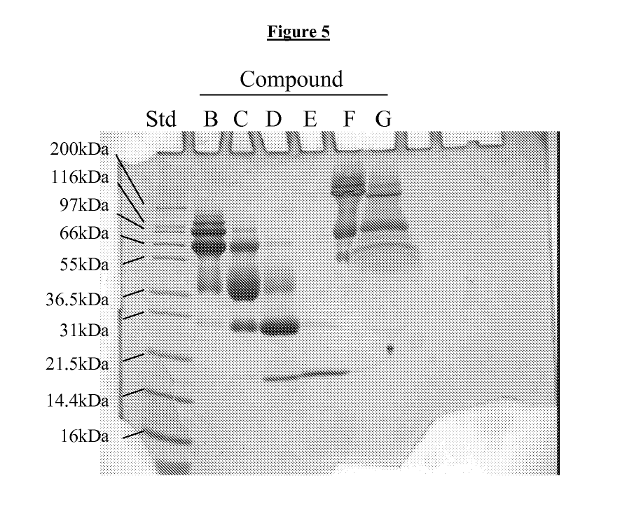

FIG. 5: SDS-PAGE analysis of final pooled products (see FIGS. 3 and 4).

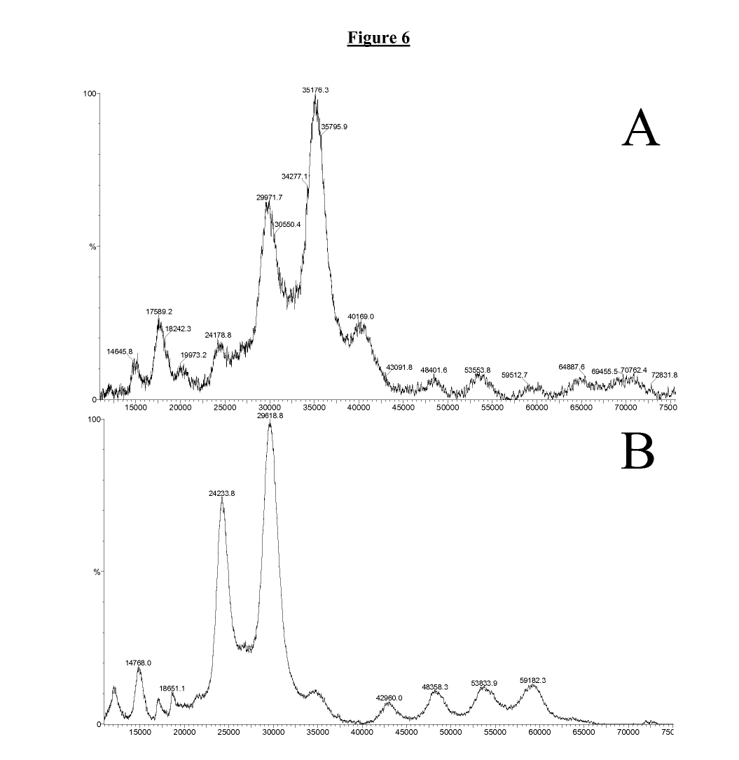

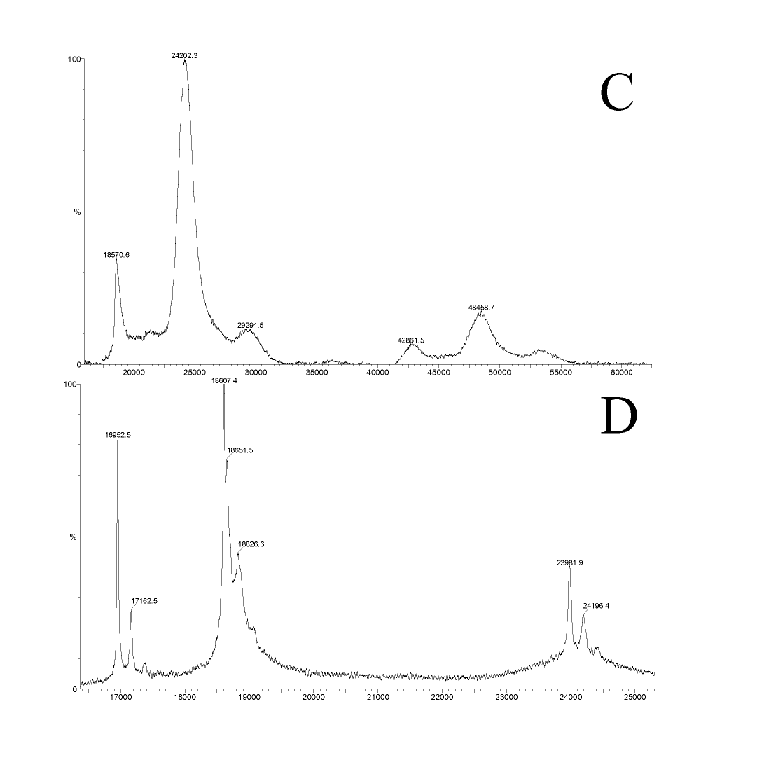

FIG. 6: Matrix-Assisted Laser Desorption Ionization-Time of Flight Mass Spectrometry (MALDI-TOF-MS)analysis of pooled fractions: (A) Compound B; (B) Compound C; (C) Compound D; (D) MR-NGE-24K, 38K, 83K, 126K.

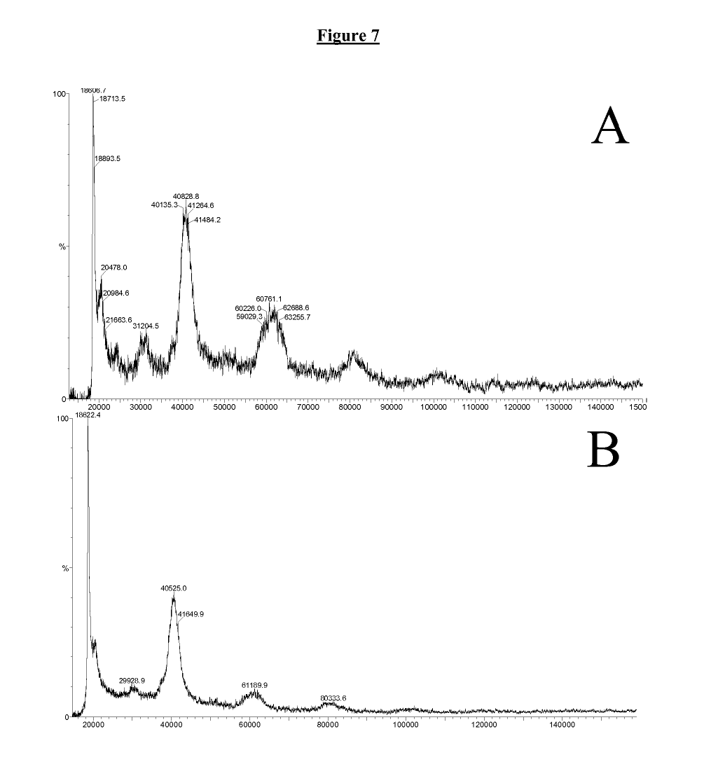

FIG. 7: MALDI-TOF-MS analysis of pooled fractions: (A) Compound F; (B) Compoud G.

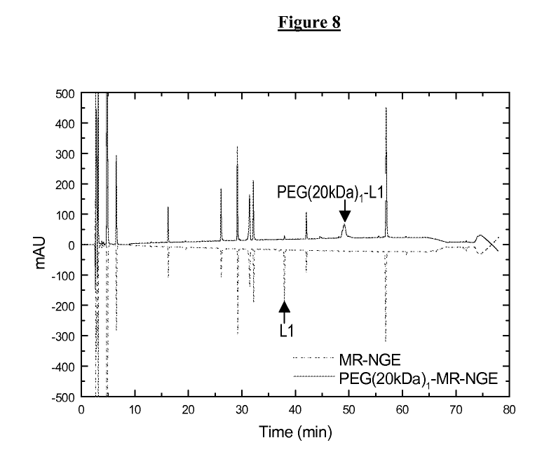

FIG. 8 Lys_C enzymatic digest of a 20 kDa monoPEGYlated-MR-NGE-166Δ (Compound Z) (Example 1e) and MR-NGE-166Δ.

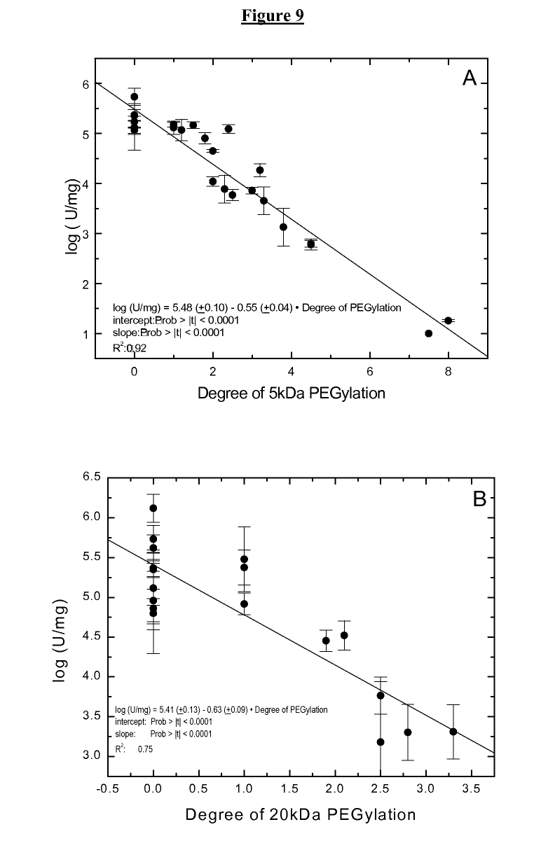

FIG. 9: Relationship between the degree of PEGylation with either 5 kDa PEG (Panel A) or 20 kDa PEG (Panel B) and in Vitro activity.

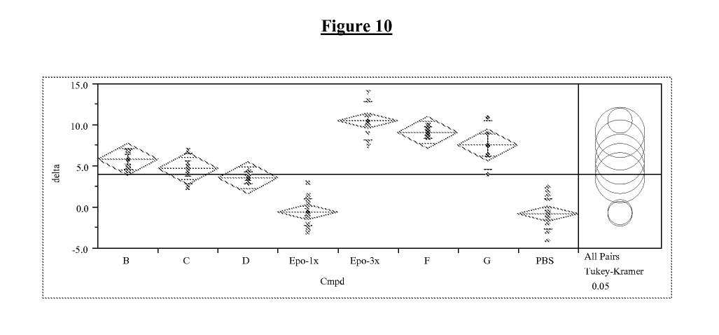

FIG. 10: In vivo activity as a function of degree of PEGylation and size of PEG moieties. Delta is the difference in hematocrit from baseline.

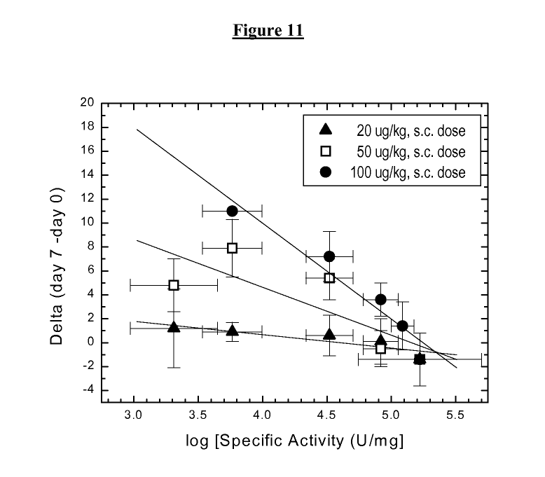

FIG. 11: Inverse correlation between in vitro and in vivo activities as a function of dose. Delta is the difference in hematocrit from baseline. The value at log[specific activity U/mg]=5.22 and Delta (day7-day0=−1.4 was estimated using the measured in vitro activity of MR-NGE-166Δ and the measured in vivo activity of PBS/BSA control.

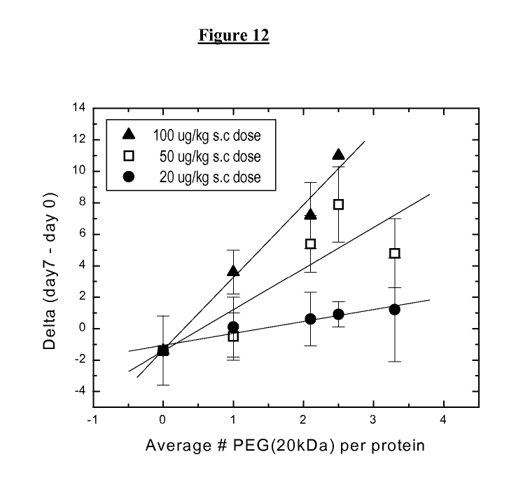

FIG. 12: In vivo activity of compound injected as a function of degree of 20 kDa pegylation. Delta is the difference between hematocrit from baseline.

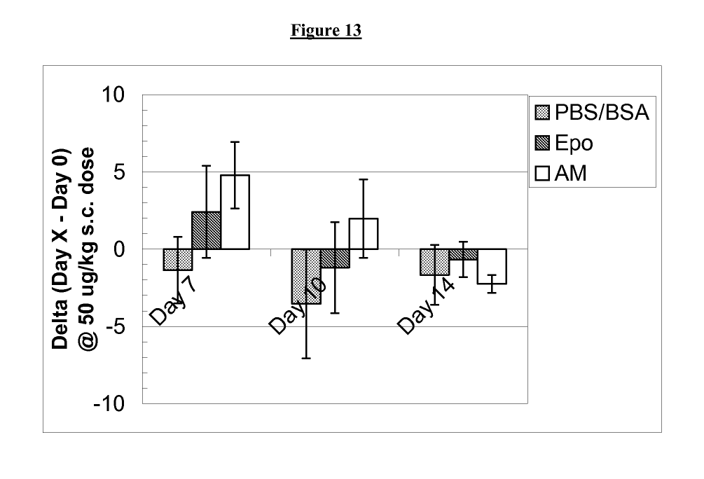

FIG. 13: In vivo activity comparison between experimental compounds and control at 7, 10, and 14 days after a single subcutaneous 50 μg/kg dose.

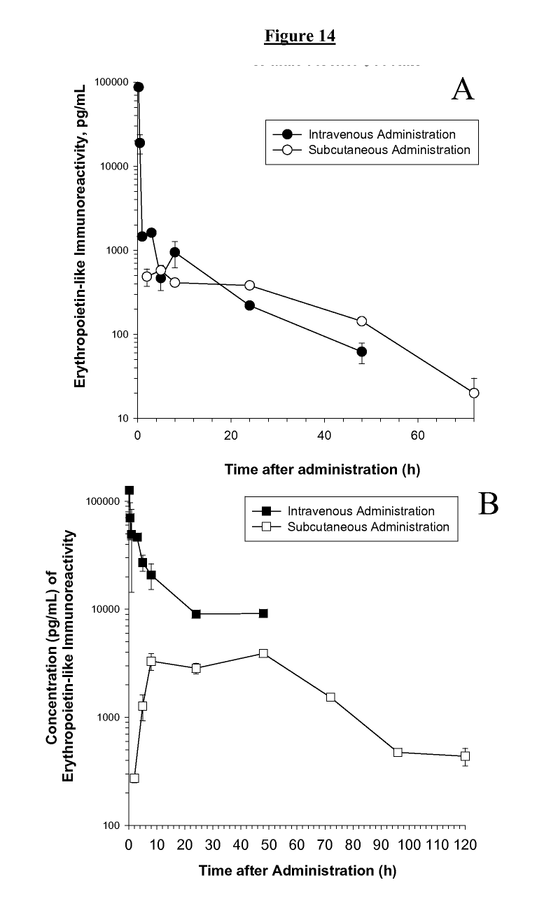

FIG. 14: Pharmacokinetic profiles of Compound C (Panel A) and Compound G (Panel B) in Fischer 344 male rats given a single dose (10 μg/kg) intravenously (solid markers) or subcutaneously (open markers).

For purposes of the present invention, as disclosed and claimed herein, the following terms and abbreviations are defined below. The terms and abbreviations used in this document have their normal meanings unless otherwise designated. For example, “° C.” refers to degrees Celsius; “mmol” refers to millimole or millimoles; “mg” refers to milligrams; “μg” refers to micrograms; “ml or mL” refers to milliliters; and “μl or μL” refers to microliters.

Amino acids abbreviations are as set forth in 37 C.F.R. § 1.822 (b)(2) (1994).

“Erythropoietin” means human erythropoietin, and is abbreviated herein as “EPO” or “huEPO.” EPO is a glycoprotein hormone that is secreted by the human kidney, that is found in human blood, and that stimulates formation of erythrocytes (erythropoiesis) in human bone marrow. The amino acid sequence of the predominant allelic variant of the protein portion of erythropoietin is known. EPO consists of 166 amino acids, is comprised of about 40% carbohydrate, by mass, and has a total molecular weight of approximately 30.4 kDa. The carbohydrate structure of EPO is heterogeneous, whereas the amino acid sequence of the predominant human allelic variant is not. Therefore, these terms refer to a heterogeneous group of EPO or huEPO molecules.

“Non-glycosylated erythropoietin” means human erythropoietin lacking attached glycosyl chains, and is abbreviated “NGE.” NGE has the amino acid sequence of EPO, but lacks N-linked glycosyl chains at positions 24, 38, and 83 and the O-linked glycosyl chain at position 126 (SEQ ID NO:4). In addition, NGE may lack the amino acid at position 166 or may have an Arg at position 166.

Non-glycosylated EPO can be conveniently expressed in cell types that lack the ability to post-translationally attach glycosyl moieties to a protein, or can be produced by enzymatically removing the glycosyl chains from EPO.

“Erythropoietin analog” means a glycosylated protein having nearly the same amino acid sequence as EPO, and having the ability to increase hematocrit when properly administered to a mammal, but differing from EPO in having one or more amino acid modifications. An amino acid modification may be an insertion, a deletion, a replacement, or an inversion of one or more amino acids. Glycosylated EPO Analogs are abbreviated “GEA.”

“Non-glycosylated erythropoietin analog” means a non-glycosylated erythropoietin having nearly the same amino acid sequence as EPO, but differing in amino sequence from EPO in having one or more amino acid modifications. Non-glycosylated erythropoietin analog is abbreviated “NGEA.”NGEAs includes non-glycosylated EPO that has the amino acid at position 166 deleted. In addition, NGEAs include EPO. wherein the amino acid-at position 166 is any amino acid.

“NGE-166Δ” represents a non-glycosylated protein having the same sequence as NGE, wherein Arg at position 166 is deleted.

““NGE-24E, 38E, 83E, 88E, 126E” and “NGE[5E])” represent a non-glycosylated protein having the same sequence as NGE, except Asn at positions 24, 38, and 83, Trp at 88, and Ser at 126 are replaced with Glu, and the Arg at position 166 is absent, present, or is any other amino acid. “NGE-24E, 38E, 83E, 88E, 126E, 166Δ” and “NGE[5E]166Δ” have the same sequence as “NGE-24E, 38E,83E, 88E, 126E” and “NGE[5E]” wherein Arg at position 166-is deleted. NGE[5E] and NGE[5E]166Δ are NGEAs.

“MR-NGE” represents a non-glycosylated protein having the same sequence as NGE, except that it has a Met-Arg leader sequence and the Arg at position 166 is absent, present, or is any other amino acid. MR-NGE-166Δ has the same sequence as MR-NGE wherein Arg at position 166 is deleted MR-NGE and MR-NGE-166Δ are NGEAs.

“MR-NGE-88E” represents a non-glycosylated protein having the same sequence as NGE, except that it has a Met-Arg leader sequence, Trp at 88 is replaced with Glu, and Arg at 166 is absent, present, or is any other amino acid. “MR-NGE-88E, 166Δ” has the same sequence as “MR-NGE-88E” wherein Arg at position 166 is deleted. MR-NGE-88E and MR-NGE-88E, 166Δare NGEAs.

“MR-NGE-88K” represents a non-glycosylated protein having the same sequence as NGE, except that it has a Met-Arg leader sequence, Trp at 88 is replaced with Lys, and Arg at position 166 is absent, present, or is any other amino acid. MR-NGE-88K, 166Δhas the same sequence as MR-NGE-88K wherein Arg at position 166 is deleted. MR-NGE-88K and MR-NGE-88K, 166Δare NGEAs.

“MR-NGE-88P” represents a non-glycosylated protein having the same sequence as NGE, except that it has a Met-Arg leader sequence, Trp at 88 is replaced with Pro, and Arg at position 166 is absent, present, or is any other amino acid. “MR-NGE-88P, 166Δ” has the same sequence as MR-NGE-88P wherein Arg at position 166 is deleted. MR-NGE-88P and MR-NGE-88P,166Δare NGEAs.

“MR-NGE-88S” represents a non-glycosylated protein having the same sequence as NGE, except that it has a Met-Arg leader sequence, Trp at 88 is replaced with Ser, and Arg at 166 is absent, present, or is any other amino acid. “MR-NGE-88S, 166Δ has the same sequence as-MR-NGE-88S wherein Arg at position 166 is deleted. MR-NGE-88S and MR-NGE-88S,166Δ are NGEAS.

“MR-NGE-76E, 88E, 139E, 154E” and “MR-NGE[4E)” represent a non-glycosylated protein having the same sequence as NGE, except that it has a Met-Arg leader sequence, Arg at 76, Trp at 88, Arg at 139,and Lys at 154 are replaced with Glu, and Arg at position 166 is absent, present, or is any other amino acid. “MR-NGE-76E, 88E, 139E, 154E, 166Δ and “MR-NGE[4E]166Δ” have the same amino acid sequence as MR-NGE-76E, 88E, 139E, 154E and MR-NGE[4E] wherein Arg at position 166 is deleted. MR-NGE[4E] and MR-NGE[4E]166Δ are NGEAs.

“MR-NGE-24E, 38E, 83E, 88E, 126E” or “MR-NGE[SE]” represents a non-glycosylated protein having the same sequence as NGE, except that it has a Met-Arg leader sequence, Asn at 24, 38, and 83, Trp at 88, and Ser at 126 are replaced with Glu, and Arg at position 166 is absent, present, or is any other amino acid. “MR-NGE-4E, 38E, 83E, 88E, 126E, 166Δ or “MR-NGE[5E]166Δ” have the same sequence as MR-NGE-24E, 38E, 83E, 88E, 126E or MR-NGE[5E] wherein Arg at position 166 is deleted. MR-NGE[5E] and MR-NGE[5E]:166Δ are NGEAs.

“MR-NGE-24K, 38K, 83K, 88K, 126K, 166Δ” or “MR-NGE[5K]” represents a non-glycosylated protein having the same sequence as NGE, except that it has a Met-Arg leader sequence, Asn at 24, 38, and 83, Trp at 88, and Ser at 126 are replaced with Lys, and Arg at position 166 is absent, present, or is any other amino acid. “MR-NGE-24K, 38K, 83K, 88K, 126K, 166Δ” or “MR-NGE[5K)166Δ” have the same sequence as MR-NGE-24K, 38K, 83K, 88K, 126K or MR-NGE[5K] wherein Arg at position 166 is deleted. MR-NGE(5K] and MR-NGE[5K]166Δare NGEAs.

“MR-NGE-24E, 38E, 83E, 126E” or “MR-NGE[W5E]” represents a non-glycosylated protein having the same sequence as NGE, except that it has a Met-Arg leader sequence, Asn at 24, 38, and 83 and Ser at 126 are replaced with Glu, and Arg at position 166 is absent, present, or is any other amino acid.

“MR-NGE-24E, 38E, 83E, 126E, 166Δ ” or MR-NGE[W5E]166Δ” have the same amino acid sequence as “MR-NGE-24E, 38E, 83E, 126E” or MR-NGE [W5E]” wherein Arg at position 166 is deleted. MR-NGE[W5E] and MR-NGE[W5E]166Δ are NGEAs.

“MR-NGE-24K, 38K, 83K, 126K” or “MR-NGE[W5K]” represents a non-glycosylated-protein-having the same sequence as NGE, except that it has a Met-Arg leader sequence, Asn at 24, 38, and 83 and Ser at 126 are replaced with Lys, and Arg at position 166 is absent, present or is any other amino acid. “MR-NGE-24K, 38K, 83K, 126K, 166Δ” or “MR-NGE[W5K]166Δ” have the same sequence as MR-NGE-24K, 38K, 83K, 126K or MR-NGE[W5K] wherein Arg at position 166 is deleted. MR-NGE[W5K) and MR-NGE[W5K]166Δare NGEAs.

Table I includes a list of NGEA compounds prepared using the cloning, expression, and purification methods described herein. Single letters represent the amino acids at particular positions in Formula I.

| | Amino acids at variable positions | | | | | | | | | | |
|---|---|---|---|---|---|---|---|---|---|---|---|
| | in Formula I | | | | | | | | | | |
| Abbreviation for NGEA | −2 | −1 | 24 | 38 | 76 | 83 | 88 | 126 | 139 | 154 | 166 |
| NGE-166Δ | — | — | N | N | R | N | W | S | R | K | — |
| NGE- | — | — | E | E | R | E | E | E | R | K | — |
| 24E, 38E, 83E, 88E, 126E, | | | | | | | | | | | |
| 166Δ | | | | | | | | | | | |
| or NGE[5E]166Δ | | | | | | | | | | | |
| MR-NGE-166Δ | M | R | N | N | R | N | W | S | R | K | — |
| MR-NGE-88E, 166Δ | M | R | N | N | R | N | E | S | R | K | — |
| MR-NGE-88K, 166Δ | M | R | N | N | R | N | K | S | R | K | — |
| MR-NGE-88P, 166Δ | M | R | N | N | R | N | P | S | R | K | — |
| MR-NGE-88S, 166Δ | M | R | N | N | R | N | S | S | R | K | — |
| MR-NGE- | M | R | N | N | E | N | E | S | E | E | — |
| 76E, 88E, 139E, 154E, 166Δ | | | | | | | | | | | |
| or MR-NGE[4E]166Δ | | | | | | | | | | | |
| MR-NGE- | M | R | E | E | R | E | E | E | R | K | — |
| 24E, 38E, 83E, 88E, 126E, | | | | | | | | | | | |
| 166Δ or MR-NGE[5E]166Δ | | | | | | | | | | | |
| MR-NGE- | M | R | K | K | R | K | K | K | R | K | — |
| 24K, 38K, 83K, 88K, 126K, | | | | | | | | | | | |
| 166Δ or MR-NGE[5K]166Δ | | | | | | | | | | | |
| MR-NGE- | M | R | E | E | R | E | W | E | R | K | — |
| 24E, 38E, 83E, 126E, 166Δ | | | | | | | | | | | |
| or | | | | | | | | | | | |
| MR-NGE[W5E]166Δ | | | | | | | | | | | |
| MR-NGE- | M | R | K | K | R | K | W | K | R | K | — |
| 24K, 38K, 83K, 126K, 166Δ | | | | | | | | | | | |
| or | | | | | | | | | | | |
| MR-NGE[W5K]166Δ | | | | | | | | | | | |

“Erythropoietic activity refers to the ability of a compound to stimulate erthyropoiesis. Erythropoietic activity can be assessed in vitro, as well as in vivo. Erythropoietic activity generally refers to the ability of a compound to cause an increase in hematocrit levels from an established base line when administered by an acceptable route of administration at effective doses. In vitro activity can be determined by the method outlined in Example 4 and in vivo activity can be determined by the method outlined in Example 5.

“Erythropoietic compound” refers to a non-glycosylated, polymer-derivatized protein having erythropoietic activity

“Polyethylene glycol” or “PEG” refers to a hydrophilic polymer having the formula:
HO(CH₂CH₂O),CH₂CH₂—OH,
wherein, x is a number from about 70 to about 1200,
preferably from about 450 to about 1200, even more
preferably from about 450 to about 700.

“PEG-aldehyde” refers to a hydrophilic polymer having the formula:
CH₃O—(CH₂CH₂O)ₓCH₂CH₂O— (CH₂)_{y}—CHO
wherein Y is number from 1 to 4, and x is a number from about 70 to about 1200, preferably from about 450 to about 1200, even more preferably from about 450 to about 700.

“PEG-Propionaldehyde” refers to a PEG-aldehyde hydrophilic polymer having the formula:
CH₃O—(CH₂CH₂O)ₓCH₂CH₂O—CH₂CH₂—CHO,
wherein, x is a number from about 70 to about 1200,
preferably from about 450 to about 1200, even more
preferably from about 450 to about 700.

“PEGylated protein” refers to a protein having 1 to 5 polymer chains of the formula: [R—O—(CH₂CH₂—O)ₓ—(CH₂)_{y}—NH], wherein R is H or C₁ to C₄ alkyl, X is a number from about 70 to about 1200, preferably from about 450 to about 1200, and even more preferably from about 450 to about 700, and Y is a number from 1 to 4; and the polymer chain is covalently bonded to the protein by a secondary amine bond.

“PEGylated NGE” refers to a non-glycosylated EPO as described above with 1 to 5 polymer chains of the formula: [R—O—(CH₂CH₂—O)ₓ—(CH₂)_{y}—NH]] wherein R is H or C₁ to C₄ alkyl, X is a number from about 70 to about 1200, preferably from about 225 to about 1200, even more preferably from about 340 to about 1200, even more preferably from about 450 to about 1200, and even more preferably from about 450 to about 700, and Y is a number from 1 to 4; and the polymer chain is covalently bonded to the protein by a secondary amine bond.

“PEGylated NGEA” refers to a non-glycosylated EPO analog as described above with 1 to 5 polymer chains of the formula: [R—O—(CH₂CH₂—O)ₓ—(CH₂)_{y}—NH], wherein R is H or C₁ to C₄ alkyl, X is a number from about 70 to about 1200,

preferably from about 225 to about 1200, even more

preferably from about 340 to about 1200, even more

preferably from about 450 to about 1200, and even more

preferably from about 450 to about 700, and Y is a number from 1 to 4; and the polymer chain is covalently bonded to the protein by a secondary amine bond.

“Mono-PEGylated NGEA” refers to a non-glycosylated EPO analog covalently attached to a single polymer chains of the formula: [R—O— (CH₂CH₂—O)ₓ—(CH₂—)_{y}—NH], wherein R is H or C₁ to C₄ alkyl, X is a number from about 70 to about 1200,

preferably from about, 225 to about 1200, even more

preferably from about 340 to about 1200, even more

preferably from about 450 to about 1200, and even more

preferably from about 450 to about 700, and Y is a number from 1 to 4; and the polymer chain is covalently bonded to the protein-by a secondary amine bond.

“Di-PEGylated NGEA” refers to a non-glycosylated EPO analog covalently attached to two polymer chains of the formula: [R—O—(CH₂CH₂—O)ₓ—(CH₂)_{y}—NH], wherein R is H or C₁ to C₄ alkyl, X is a number from about 70 to about 1200,

preferably from about 225 to about 1200, even more

preferably from about 340 to about 1200, even more

preferably from about 450 to about 1200, and even more

preferably from about 450 to about 700, and Y is a number

from 1 to 4; and the polymer chain is covalently bonded to the protein by a secondary amine bond.

“Tri-PEGylated NGEA” refers to a non-glycosylated EPO analog covalently attached to three polymer chains of the formula: [R—O— (CH₂CH₂—O)ₓ— (CH₂)_{y}—NH], wherein R is H or C₁ to C₄ alkyl, X is a number from about 70 to about 1200,

preferably from about 225 to about.1200, even more

preferably from about 340 to about 1200, even more

preferably from about 450 to about 1200, and even more

preferably from about 450 to about 700, and Y is a number from 1 to 4; and the polymer chain is covalently bonded to the protein by a secondary amine bond.

“Tetra-PEGylated NGEA” refers to a non-glycosylated EPO analog covalently attached to four polymer chains of the formula: [R—O—(CH₂CH₂—O)ₓ—(CH₂)_{y}—NH], wherein R is H or C₁ to C₄ alkyl, X is a number from about 70 to about 1200,

preferably from about 225 to about 1200, even more

preferably from about 340 to about 1200, even more

preferably from about 450 to about 1200, and even more

preferably from about 450 to about 700, and Y is a number from 1 to 4; and the polymer chain is covalently bonded to the protein by a secondary amine bond.

“Multi-PEGylated NGEA” refers to a non-glycosylated EPO analog covalently attached to four or more polymer chains of the formula: [R—O—(CH₂CH₂—O)ₓ—(CH₂)_{y}—NH], wherein R is H or C₁ to C₄ alkyl, X is a number from about 70 to about 1200,

preferably from about 225 to about 1200, even more

preferably from about 340 to about 1200, even more

preferably from about 450 to about 1200, and even more

preferably from about 450 to about 700, and Y is a number from 1 to 4; and the polymer chain is covalently bonded to the protein by a secondary amine bond.

“Lys_C” refers to an endoprotease with specificity for cleaving peptides and proteins C-terminal to lysine residues.

“All nucleic acid sequences, unless otherwise designated, are written in the direction from the 5′ end to the 3′ end, frequently referred to as “5′ to 3′.”

All amino acid or protein sequeences, unless otherwise designated, are written commencing with the amino-terminus (“N-terminus”) and “concluding with the carboxy-terminus (“C-terminus”).

“Base, pair” or “bp” as used herein refers to DNA or RNA. The abbreviations A, C, G, and T correspond to the 5′-monphosphate forms of the deoxyribonucleosides (deoxy)adenosine, (deoxy)cytidine, (deoxy)guanosine, and thymidine, respectively, when they occur in DNA molecules. The abbreviations U, C, G and A correspond to the 5′-mmonophosphate forms of the ribonucleosides uridine, cytidine, guanosine, and adenosine, respectively when they occur in RNA molecules. In double stranded DNA, base pair may refer to a partnership of A with T or C with G. In a DNA/RNA, heteroduplex base pair may refer to a partnership of A with U or C with G. (See the definition of “complementary” infra.)

“Digestion” or “Restriction” of DNA refers to the catalytic cleavage of the DNA with a restriction enzyme that acts only at certain sequences in the DNA (“sequence-specific endonucleases”). The various restriction enzymes used herein are commercially available and their reaction conditions, cofactors, and other requirements were used as would be known to one of ordinary skill in the art. Appropriate buffers and substrate amounts for particular restriction enzymes are specified by the manufacturer or can be readily found in the literature.

“Ligation” refers to the process of forming phosphodiester bonds between two double stranded nucleic acid fragments. Unless otherwise provided, ligation may be accomplished using known buffers and conditions with a DNA ligase, such as T4 DNA ligase.

“Plasmid” refers to an extrachromosomal (usually) self-replicating genetic element. Plasmids are generally designated by a lower case “p” followed by letters and/or numbers. The starting plasmids herein are either commercially available, publicly available on an unrestricted basis, or can be constructed from available plasmids in accordance with published procedures. In addition, equivalent plasmids to those described are known in the art and will be apparent to the ordinarily skilled artisan.

“Recombinant DNA cloning vector[ as used herein refers to any autonomously replicating agent, including, but not limited to, plasmids and phages, comprising a DNA-molecule to which one or more additional DNA segments can or have been added.

“Recombinant DNA expression vector” as used herein refers to any recombinant DNA cloning vector in which a promoter to control transcription of the inserted DNA has been incorporated.

“Transcription” refers to the process whereby information contained in a nucleotide sequence of DNA is transferred to a complementary RNA sequence.

“Transfection” refers to the uptake of an expression vector by a host cell whether or not any coding sequences are, in fact, expressed. Numerous methods of transfection are known to the ordinarily skilled artisan, for example, calcium phosphate co-precipitation, and electroporation. Successful transfection is generally recognized when any indication of the operation of this vector occurs within the host cell.

“Transformation” refers to the introduction of DNA into an organism so that the DNA, is replicable, either as an extrachromosomal element or by chromosomal integration. Methods of transforming bacterial and eukaryotic hosts are well known in the art, many of which methods, such as nuclear injection, protoplast fusion or by calcium treatment using calcium chloride are summarized in J. Sambrook, et al., Molecular Cloning: A Laboratory Manual, (1989).

Generally, when introducing DNA into Yeast the term transformation is used as opposed to the term transfection.

“Translation” as used herein refers to the process whereby the genetic information of messenger RNA (mRNA) is used to specify and direct the synthesis of a polypeptide chain.

“Vector” refers-to a nucleic acid compound used for the transfection and/or transformation of cells in gene manipulation bearing polynucleotide sequences corresponding to appropriate protein molecules which, when combined with appropriate control sequences, confers specific properties on the host cell to be transfected and/or transformed. Plasmids, viruses and bacteriophages are suitable vectors. Artificial vectors are constructed by cutting and joining DNA molecules from different sources using restriction enzymes and ligases. The term “vector” as used herein includes Recombinant DNA cloning vectors and Recombinant DNA expression vectors.

“Complementary” or “Complementarity”, as used herein, refers to pairs of bases (purines and pyrimidines) that associate through hydrogen bonding in a double stranded nucleic acid. The following base pairs are complementary: guanine and cytosine; adenine and thymine; and adenine and uracil.

“Hybridization” as used herein refers to a process in which a strand of nucleic acid joins with a complementary strand-through base pairing. The conditions employed in the hybridization of two non-identical, but very similar, complementary nucleic acids varies with the degree of complementarity of the two strands and the length of the strands. Such techniques and conditions are well known to practitioners in this field.

“Isolated amino acid sequence” refers to any amino acid sequence, however, constructed or synthesized, which is locationally distinct from the naturally occurring sequence.

“Isolated DNA compound” refers to any DNA sequence, however constructed or synthesized, which is Vocationally distinct from its natural location in genomic DNA.

“Isolated nucleic acid compound” refers to any RNA or DNA sequence, however constructed or synthesized, which is locationally distinct from its natural location.

“Primer” refers to a nucleic acid fragment which functions as an initiating substrate for enzymatic or synthetic elongation.

“Promoter” refers to a DNA sequence which directs transcription of DNA to RNA.

“Probe” refers to a nucleic acid compound or a fragment, thereof, which hybridizes with another nucleic acid compound.

“stringency” refers to a set of hybridization conditions which may be varied in order to vary the degree of nucleic acid affinity for other nucleic acids.

“PCR” refers to the widely-known polymerase chain reaction employing a thermally-stable DNA polymerase.

“Leader sequence” refers to an N-terminal sequence of amino acids which can be enzymatically or chemically removed to produce the desired polypeptide of interest.

“Secretion signal sequence” refers to a sequence of amino acids generally present at the N terminal region of a larger polypeptide functioning to initiate association of that polypeptide with the cell membrane and secretion of that polypeptide through the cell membrane.

This invention provides derivatives of non-glycosylated EPO and non-glycosylated EPO analogs which have improved therapeutic properties compared to glycosylated EPO proteins. The NGE and NGEA derivatives of the present invention are produced by expression in a recombinant system followed by modification with polyethylene glycol (PEG).

Non-tderivitized non-glycosylated EPO proteins of the present invention do not have practical in vivo activity. [Lin, et al., U.S. Pat. No. 4,703,008; K. Yamaguchi, et al., (199,1) Effects of site-directed removal of N-glycosylation sites in human erythropoietin on its production and bi logical properties, J. of Biol. Chem. 266:20434-20439]. However, PEGylation of non-glycosylated EPO and certain non-glycosylated EPO analogs imparts properties such as increased plasma half-life, reduced immunogenicity and antigenicity, improved solubility, reduced proteolytic susceptibility,improved hioavailability, reduced toxicity, reduced affinity to serum binding proteins, improved thermal and mechanical stability, as well as, improved compatibility with depot formulations compared to glycbsylated erythropoietin and NGE.

Thus, it is an object of the present invention to produce polymer derivatized non-glycosylated erythropoietic compounds with improved properties compared with EPO, and thereby avoiding the perceived problems associated with increasing the carbohydrate content of EPO. These problems include reduce bioavailability and unfavorable pharmacokinetics.

Preparation of NGE and NGEAs:

The compounds of the present invention may be produced by a variety of methods including recombinant DNA technology or well known chemical procedures, such as solution or solid-phase peptide synthesis, or semi-synthesis in solution beginning with protein fragments coupled through conventional solution methods.

Vectors and Host Cells:

The present invention also relates to vectors that, include isolated nucleic acid molecules, host cells that are genetically engineered with the recombinant vectors, and the production of NGE or NGEAs by recombinant techniques.

The nucleotides encoding the proteins of the present invention can optionally be joined to a vector containing a selectable marker for propagation in a host. Generally, with respect to mammalian cell hosts, a plasmid vector is introduced in a precipitate, such as a calcium phosphate precipitate, or in a complex with a charged lipid, or by other methods that are well known to those with ordinary skill in the art. If the vector is a viral vector, it can be introduced directly into mammalian host cells or introduced using viral supernatant produced by packaging in vitro using an appropriate packaging cell line. Bacterial viral vectors (bacteriophages) can also be packaged in vitro using packaging cell extracts commercially available and then transfected into host bacterial cells.

The DNA insert should be operatively linked to an appropriate promoter, such as the phage lambda PL promoter, the E. coli lac, trp and tac promoters, the SV40 early and late promoters and promoters of retroviral LTRs, as well as the glyceraldehyde phosphate dehydrogenase (GAPDH) and alcohol oxidase (AOX) promoters to name a few. Other suitable promoters will be known to the skilled artisan. The expression constructs will further contain sites for transcription initiation, termination and, in the transcribed region, a ribosbme binding site for translation.

The coding portion of the mature transcripts expressed by the constructs will preferably include a translation initiating at the beginning and a termination codon (e.g., UAA, UGA or UAG) appropriately positioned at the end of the mRNA to be translated.

Expression vectors will preferably include at least one selectable marker. Such markers include, e.g., dihydrofolate redudtase or neomycin resistance for mammalian cell culture, neomycin resistance or complementation of auxotrophid markers for yeasts, and tetracycline, ampicillin, kanamycin, or chloramphenicol resistance genes for culturing in E. coli and other bacteria. Representative examples of appropriate hosts include, but are not limited to, bacterial cells, such as E. coli, Streptomyces and Salmonella typhimurium cells; fungal cells, such as Aspergillus niger; yeast cells, such as Pichia pastorisand Saccharomyces cerevisiae; insect cells such as Drosophila S2 and Spodoptera Sf9 cells; animal cells such as CHO, COS, AV-12, HEK293, and Bowes melanoma cells; and plant cells such as tobacco, corn, and soybean. Appropriate culture mediums and conditions for the above-described host cells are known in the art. Vectors preferred for use in bacteria include pQE70,pQE60 and pQE-9, available from Qiagen; pBS vectors, Phagescript vectors, Bluescript vectors, PNH8A, pNH16a, pNH18A, pNH46A, available from Stratagene; pET30 vectors from Novagen, and ptrc99a, pKK223-3, pKK233-3, pDR540, pRIT5 available: from Pharmacia. Preferred eucaryotic vectors include pWLNEO, pSV2CAT, pOG44, pXT1 and pSG available from Stratagene; pcDNA3, pRcRSV, and pRcCMV from Invitrogen, Inc.; and pSVK3, PBPV, pMSG and pSVL available from Pharmacia. Preferred vectors-for expression in Pichia pastoris include the pPIC vectors commercially available from Invitrogen, Inc. Other suitable vectors will be readily apparent to the skilled artisan.

Introduction of a vector construct into a host cell can be effected by calcium phosphate transfection, DEAE-dextran mediated transfection, cationic lipid-mediated transfection, electroporation, transduction, infection, transformation or other methods. Such methods are described in many standard laboratory manuals, such as Ausubel, et al., ed., Current Protocols in Molecular Biology, Greene Publishing, NY, N.Y. (1987-1998) and Sambrook, et al., Molecular Cloning: A Laboratory Manual, 2^{nd} Edition, Cold Spring Harbor, N.Y. (1989).

The proteins of the present invention can be expressed in a modified form, such as a fusion protein, and can include not only secretion signals, but also additional heterologous functional regions. For instance, a region of additional amino acids can be added to the N-terminus of an analog to improve stability and persistence in the host cell, during purification, or during subsequent handling and storage. Also, peptide moieties can be added to facilitate purification. Such regions can be removed prior to final preparation of an active protein. Such methods are described in many standard laboratory manuals, such as Sambrook, supra, Chapters 17.29-17.42 and 18.1-18.74; Ausubel, supra, Chapters 16, 17 and 18.

Specifically, a gene encoding MR-NGE-166Δ was constructed synthetically by in vitro hybridization using a set of six overlapping oligonucleotides from the positive strand of human erythropoietin cDNA with six complementary oligonucleotides (negative strand). The codon usage for the synthetic MR-NGE-166Δ gene was 100% optimized for E. coli codon usage (Wisconsin Package, v.8) while maintaining a low GC content at the 5′ end. The hybridized oligonucleotides were ligated with T4 DNA ligase and the ligation product amplified by PCR using Pfu turbo DNA polymerase (Strategene).

PCR introduced two restriction endonuclease cleavage sites into the synthetic MR-NGE-166Δ gene, a 5′ NdeI site and a 3′ BamHI site. The MR-NGE-166ΔPCR product was then cloned into the NdeI and BamH I sites in-the commercial expression vector pET30a (Novagen) to create pET30a_MR-NGE-166Δ (FIG. 1). Protein expression was carried out using the commercial expression strain BL21(DE3) (Novagen). Genes encoding non-glycosylated erythropoietin analogs were prepared by introducing point mutations at different positions using PCR according to the procedures described by Nelson, R., M., and Long, G., L., (1989), Anal. Biochem. 180:147-151.

Expression of Proteins in Host Cells

Using nucleic acids of the present invention, one may express a protein of the present invention in a recombinantly engineered cell, such as bacteria, yeast, insect, or mammalian cells. The cells produce the protein in a non-natural condition (e.g., in quantity, composition, location, and/or time), because they have been genetically altered through human intervention to do so.

It is expected that those of skill in the art are knowledgeable in the numerous expression systems available for expression of a nucleic acid encoding a protein of the present invention. No attempt to describe in detail the various methods known for the expression of proteins in prokaryotes or eukaryotes will be made.

In brief summary, the expression of isolated nucleic acids encoding a protein of the present invention will typically be achieved by operably linking, for example, the DNA or cDNA to a promoter (which is either constitutive or inducible), followed by incorporation-into an expression vector. The vectors can be suitable for replication and integration in either prokaryotes or eukaryotes. Typical expression vectors contain transcription and translation terminators, initiation sequences and promoters useful for regulation of the expression of the DNA encoding a protein of the present invention. To obtain high level expression of a cloned gene, it is desirable to construct expression vectors which contain, at the minimum, a strong promoter to direct transcription, a ribosome binding site for translational initiation, and a transcription/translation terminator.

Alternatively, nucleic acids of the present invention may be fused downstream of inducible promoters. Gene expression is then induced by exposing a host cell containing the gene of interest fused downstream of the inducible promoter to a specific transcriptional inducer. Such methods are well known in the art, e.g., as described in U.S. Pat. Nos. 5,580,734, 5,641,670, 5,733,746, and 5,733,761, entirely incorporated herein by reference.

Expression in Prokaryotes

Prokaryotic cells may be used as hosts for expression. Prokaryotes most frequently are represented by various strains of E. coli; however, other microbial strains may also be used. Commonly used prokaryotic control sequences which are defined herein to include promoters for transcription initiation, optionally with an operator, along with ribosome binding site sequences, include such commonly used promoters as the beta lactamase (penicillinase) and lactose (lac) promoter systems (Chang, et al., Nature 198:1056 (1977)), the tryptophan (trp) promoter system (Goeddel, et al., Nucleic Acids Res. 8,:4057 (1980)), the bacteriaphage T7 promoter and RNA polymerase, and the bacteriaphage lambda derived P L promoter and N-gene ribosome binding site (Shimatake, et al., Nature 292:128 (1981)). The inclusion of selection markers in DNA vectors transfected in E. coli is also useful.

Examples of such markers include genes specifying resistance to ampicillin, tetracycline, kanamydin, or chloramphenicol.

The vector is selected to allow introduction into the appropriate host cell. Bacterial vectors are typically of plasmid or phage origin. Appropriate bacterial cells are infected with phage vector particles or transfected with naked phage vector DNA. If a plasmid vector is used, the bacterial cells are transformed with the plasmid vector DNA. Expression systems for expressing a protein of the present invention are also available using Bacillus subtilis and Salmonella (Palva, et al., Gene 22:229-235 (1983); Mosbach, et al., Nature 302:54,3-545 (1983)).

For example, pET30a MR-NGE-166Δ was transfected into the commercial expression strain, BL21(DE3) (Novagen) (FIG. 1). Log phase, cells were typically grown at 37° C. to OD₆₀₀=0.9 and induced to express MR-NGE-166Δ with 1 mM IPTG and harvested 3 hours post-induction. The above fermentation conditions resulted in high expression levels of MR-NGE-166Δ (>100 mg/L) that accumulated in inclusion bodies.

Expression in Eukaryotes

A variety of eukaryptic expression systems such as yeast, insect cell lines, plant and mammalian cells, are known to those of skill in the art. As explained briefly below, a nucleic acid of the present invention can be expressed in these eukaryotic systems.

Synthesis of heterologous proteins in yeast is well known. F. Sherman, et al., Methods in Yeast Genetics, Cold Spring Harbor Laboratory (1982) is a well-recognized work describing the various methods available to produce the protein in yeast. Two widely utilized yeast systems for production of eukaryotic proteins are Saccharomyces cerevisiae and Pichia pastoris. Vectors, strains, and protocols for expression in Saccharomyces and Pichia are known in the art and available from commercial suppliers (e.g., Invitrogen). Suitable vectors usually have expression control sequences, such as promoters, including 3-phosphoglycerate kinase or alcohol oxidase, and an origin of replication, termination sequences and the like as desired.

The sequences encoding proteins of the present invention can also be ligated to various expression vectors for use in transfecting cell cultures of, for instance, mammalian, insect, or plant origin. Illustrative of cell cultures useful for the production of the peptides are mammalian cells. Mammalian cell systems often will be in the form of monolayers of cells although mammalian cell suspensions may also be used. A number of suitable host-cell lines capable of expressing intact proteins have been developed in the art, and include the HEK293, BHK21, AV-12, and CHO cell lines. Expression vectors for these cells can include expression control sequences, such as an origin of replication, a promoter (e.g., the CMV promoter, a HSV tk promoter or pgk (phosphoglycerate kinase) promoter), an enhancer (Queen, et al., Immunol. Rev. 89:49 (1986)), and processing information sites, such as ribosome binding sites, RNA splice sites, polyadenylation sites (e.g., SV40 large T Ag poly A addition site or Bovine growth hormone poly A addition site), and transcriptional terminator sequences. Other animal cells useful for production of proteins of the present invention are available, for instance, from the American Type Culture Collection Catalogue of Cell Lines and Hybridomas (7th edition, 1992).

Appropriate vectors for expressing proteins of the present invention in insect cells are usually derived from the SF9 baculovirus. Suitable insect cell lines include mosquito larvae, silkworm, armyworm, moth and Drosophila cell lines such as a Schneider cell line (See Schneider, J. Embryol. Exp. Morphol. 27:353,-365 (1987).

As with yeast, when higher animal or plant host cells are employed, polyadenylation or transcription terminator sequences are typically incorporated into the vector. An example of a terminator sequence is the polyadenlyation sequence from the bovine growth hormone gene. Sequences for accurate splicing of the transcript may also be included. An example of a splicing sequence is the VP1 intron from SV40 (Sprague, et al., J. Virol. 45:773-781 (1983)). Additionally, gene sequences to control replication in the host cell may be incorporated into the vector such as those found in bovine papilloma virus type-vectors. M. Saveria-Campo, Bovine Papilloma Virus DNA, a Eukaryotic Cloning Vector in DNA Cloning Vol. II, a Practical Approach, D. M. Glover, Ed., IRL Press, Arlington, Va., pp. 213-238 (1985).

Signal Peptides:

Signal peptides may be used to facilitate the extracellular discharge of proteins in both prokaryotic and eukarybtic environments. It has been shown that the addition of a heterologous signal peptide to a normally cytosolic protein may result in the extracellular transport of the normally cytosolic protein. Alternate signal peptide sequences may function with heterologous coding sequences.

Signal peptides, such as the 27 amino acid human EPO secretion signal sequence, the alpha factor peptide or the human serum albumin signal peptide can be incorporated into the: modified EPO proteins of the present invention to facilitate extracellular translocation or intracellular destination.

Leader Sequences:

The present invention contemplates leader sequences having a variable sequence of amino acids fused to the N-terminus of the mature protein. The, leader sequence is preferably exposed to the solvent which enables it to be cleaved by DAP or other aminopeptidases, e.g., the cathepsins. A leader sequence consisting of amino acids which will facilitate exposure of the leader to the solvent and allow for subsequent removal by DAP is preferred. A. preferred leader sequence will contain an even number from two to twenty hydrophilic amino acids which can be cleaved by a specific enzyme such as a serine protease or a DAP enzyme. A more preferred leader sequence is the sequence Met-Arg fused to the N-terminal amino acid of the NGEAs of the present invention (see Table I).

Protein Folding:

Once an expression vector carrying a gene encoding a NGE or NGEA of the present invention is transfected into a suitable host cell using standard methods, cells that contain the vector are propagated under conditions suitable for expression of the recombinant analog protein. For example, if the recombinant gene has been placed under the control of an inducible promoter, suitable growth conditions would incorporate the appropriate inducer. The recombinantly produced protein may be purified from cellular extracts of transformed cells by any suitable means. Preferably, the E. coli, derived inclusion bodies are solubilized using a denaturing solution, preferably containing urea or guanadine hydrochoride, and protein initially purified by cation or anion exchange chromatography, depending on the pI of the protein being purified. Preferably, the eluted protein can be refolded by exhaustive dialysis against a renaturing solution or by infinite dilution which involves dripping the protein containing solution into a renaturing buffer. The refolded protein can then be concentrated using either a tangential flow-filtration system with a S3Y10 spiral cartridge, Amicon Flow through concentrator, or PEG-induced dehydration in a dialysis bag.

Protein Purification:

NGE and NGEAs of the present invention can be purified from recombinant cell cultures by well-known methods including ammonium sulfate or ethanol precipitation, acid extraction, anion or cation exchange chromatography, phosphocellulose chromatography, hydrophobic interaction chromatography, affinity chromatography, reversed-phase chromatography, hydroxylapatite chromatography, and size exclusion chromatography. Most preferably anion or cation exchange chromatography, phbsphocellulose chromatography and size exclusion chromatography are used.

Additionally, the non-glycosylated EPO proteins of the present invention may be used at the N-terminal or C-terminal end to several histidine residues. This “histidine-tag” enables a single-step protein purification method referred to as “immobilized metal ion affinity chromatography” (IMAC), essentially as described in U.S. Pat. No. 4,569,794, which hereby is incorporated by reference.

The IMAC method enables rapid isolation of substantially pure protein starting from a crude extract of cells that express a recombinant protein, as described above.

Cleavage of Leader Sequences:

Following expression of non-glycosylated EPO proteins containing N-terminal leader sequences in bacteria, yeast, or higher eukaryotic cells, the proteins can be digested with an aminopeptidase such as a mono- or di-aminopeptidase, a serine protease such as trypsin, or even by chemical cleavage such as cleavage by cyanogen bromide. Watson, et al., (1976) Methods Microb. 9:1-14 describe different aminopeptidases present in different bacteria including E. coli and is entirely herein incorporated by reference.

Dipeptidylaminopeptidases (DAPs) are enzymes which hydrolyze the penultimate amino terminal peptide bond releasing dipeptides from the unblocked amino-termini of peptides and proteins. There are currently four classes of dipeptidylaminopeptidases (designated DAP-I, DAP-II, DAP-III and DAP-IV) that differ based on their physical characteristics and the rates at which they react with their substrates. DAP-I is a relatively non-specific DAP that catalyzes the release of many dipeptide combinations from the unblocked amino termini of peptides and proteins. DAP-I shows little or no activity if the emergent dipeptide is Pro-X, or X-Pro (where X is any amino acid). DAP-II shows a preference for amino terminal dipeptide sequences that begin with Arg-X or Lys-X, and to a lesser extent, X-Pro. DAP-II exhibits significantly lower reaction rates versus most other dipeptide combinations. DAP-III appears to have a propensity toward amino terminal dipeptide sequences of the form Arg-Arg and Lys-Lys. DAP-IV shows its highest rate of hydrolytic activity toward dipeptide sequences of the form X-Pro. The DAP enzymes, particularly DAP-I and DAP-IV, have been shown to be useful in processing proteins.

Processing of percursor polypeptides containing leader sequences by bovine dipeptidylaminopeptidase is disclosed in Becker et al., U.S. Pat. No. 5,126,249 and is herein incorporated by reference. One particular DAP commonly used to process precursor polypeptides is that derived from the slime mold Dictyostelium discoidium. The synthesis, purification and use of this protease, often abbreviated as dDAP, are described in European Patent Publication 595,476, published May 4, 1994, and U.S. patent application Ser. Nos. 08/301,519, filed Sep. 7, 1994, and 08/445,308, filed May 19, 1995, all of with are herein incorporated by reference.

The dDAP reaction is generally conducted in an aqueous medium suitably buffered to obtain and maintain a pH from about 2.0 to about 6.5. Preferably, the pH of the medium ranges from about 3.0 to about 4.5, and most preferably, from about 3.0 to about 3.5. The dDAP reaction, however, may be conducted at a pH higher than 6.5 in the presence of urea.

Characterization of Non-Glycosylate EPO Analogs:

The development of non-glycosylated proteins having in vivo erythropoietic activity has been difficult due to the limited solubility and stability of the proteins in the absence of their native carbohydrate chains. [Owers-Narhi, et al., (1991) The effect of carbohydrate on the structure and stability of erythropoietin, J. Biol. Chem. 266:23022-23026]. The NGEs and NGEAs of the present invention, however, have been successfully cloned, expressed in E. coli inclusion bodies, refolded, and purified.

NGE and NGEAs generally have in vitro activity that is comparable to glycosylated human EPO. Yet, as discussed briefly above, NGE and NGEAs do not have any detectable in vivo activity, i.e., no increase in hematocrit was observed, in 6 week old B6C3F1 mice dosed at 0.2 μg/mouse/injection with 4 injections/day, for two weeks. This lack of in vivo activity is likely due to the rapid clearance of the molecule. The rapid clearance is hypothesized to be associated with either receptor-based and/or renal clearance mechanisms. However, surface partitioning to cell surface glucosaminoglycans or binding to circulating binding-proteins cannot be excluded. Rapid clearance of non-glycosylated EPO was demonstrated by the 150-fold reduction in plasma levels observed within 30 minutes after an intravenous dose of a NGEA (e.g. MR-NGE-166Δ) (FIG. 2). The subcutaneous dose showed analogous reduction in plasma levels (FIG. 2).

Thus, whether a particular compound stimulates in vivo erythropoiesis cannot be accurately predicted based solely on its in vitro activity. NGE and NGEAs are highly active in vitro, yet show minimal or no in vivo activity. In addition, PEGylated EPO analog species can have reduced in vitro activity, yet have enhanced in vivo activity. In vitro activity, however, is useful as a general indicator of the ability of the molecule to bind to the receptor and induce a response. Thus, in vitro activity provides some information regarding whether a particular mutation will have positive or negative effects on activity. Many NGEA derivitives, such as mono- and di-PEGylated NGEAs, have in vitro activities similar to a commercial GEA. However, these molecules exhibit varying degrees of in vivo activity compared with a commercial GEA.

Furthermore, the pharmacokinetic results show that the size of the polymer is critical for bioavailability. Surprisingly, a NGEA derivatized with 20 kDa PEG had greater subcutaneous bioavailability than one derivatized with 5 kDa PEG. This was highly unexpected.

Physical stability is also essential for therapeutic formulations. The physical stability of NGE and NGEA depends on their conformational stability, charge residues at one or more of the glycosylation sites, ionic strength, pH, protein concentration, and glycerol concentration, among other possible factors. MR-NGE[5E], MR-NGE[5K], MR-NGE[W5E], and MR-NGE[W5K) have similar or improved conformational and physical stability relative to NGE at 0.1 mg/mL protein in 20 mM TRIS, 0.5 M NaCl, 1 mM EDTA, pH 7.4, 37° C. as measured in unfolding studies by circular dichroism and aggregation studies by 90° light scattering measurements, respectively. It has been also identified that the physical stability of these proteins can be modulated by ionic strength and glycerol. Surprisingly, the non-glycosylated EPO analogs of the present invention show enhanced physical stability at NaCl ranging from 150 mM to 1 M. Furthermore, the physical stability of proteins were enhanced with increasing amounts of glycerol, 0-35%. The improved solubility and physical stability of these non-glycosylated molecules are beneficial during their purification and derivatization.

Stability studies indicate that point mutations at position 88 alone have a deleterious effect on physical stability regardless of the charge introduced at this site. However, this reduction in physical stability can be mitigated by the introduction of negative or positive charges at the glycosylation sites. Physical stability and conformational studies indicate that improved stability in these analogs is due to a convolution of effects that include conformational stability and surface charge affects. However, it should be recognized that physically unstable agonists created by single point mutations at position 88 can be derivatized to create molecules that exhibit the in vivo activity and physical stability needed for a practical therapeutic.

PEGylation of Non-Glycosylated EPO and Non-Glycosylated EPO Analogs:

Once the non-glycosylated EPO proteins of the present invention are appropriately expressed, refolded (depending on the expression system used), and purified, they can be modified. Proteins can be modified by covalently linking synthetic or natural macromolecules to the surface of the proteins. However, it has been difficult to endow delicate proteins with suitable new properties by attaching polymers without causing any loss of their functionality.

The present invention provides specific NGE and NGEA derivatives which are modified by polyethylene glycol. A wide variety of methods have been developed to produce proteins modified by polyethylene glycol (PEG). PEGylation of proteins can overcome many of the pharmacological and toxicological problems associated with using proteins as therapeutics. However, for any individual protein it is uncertain whether modification by polyalkylene groups will cause significant losses in bioactivity.

The bioactivity of polymer modified proteins can be effected by factors such as: i) the size of the polymer; ii) the particular sites of attachment; iii) the degree of modification; iv) adverse coupling conditions; v) whether a linker is used for attachment or whether the polymer is directly attached; vi) generation of harmful co-products; vii) damage inflicted by the activated polymer; or viii) retention of charge. Depending on the coupling reaction used, polymer modification of cytokines, in particular, has resulted indramatic reductions in bioactivity. Francis, G. E., et al., (1998) PEGylation of cytokines and other therapeutic proteins and peptides: the importance of biological optimization of coupling techniques, Intl. J. Hem. 68:1-18.

The present invention provides non-glycosylated EPO and non-glycosylated EPO analogs with polyethyleneglycol polymers covalently attached, thereto. The methods of this invention are used to directly attach polymers which vary in size. Furthermore, the addition of polymers is controlled such that a bioactive population of NGE protein derivatives can be purified for therapeutic use.

There are numerous methods of covalently attaching polyalkylenne polymers to proteins. For example, Inada, et al., describe a method using cyanuric chloride, 2,4,6-trichloro-s-triazine and monomethoxypolyethylene glycol. Inada, et al., (1986) Engineering physicochemical and biological properties of protein by chemical modification, Trends, Biotech. March:68-73. The preferred method for preparing the NGE protein derivative's of the present invention, however, involves the use of Polyethylene glycol-propiqnaldehyde (PEG-propionaldehyde) or polyethylene glycol-acetaldehyde (PEG-acetaldehyde) to directly attach ethylene glycol groups to amino groups. The amino groups include the N-terminus and lysine residues.

The PEGylation process of the present invention utilizes a stable linker less aldehyde modification process, via reductive alkylation. This method minimizes immunogenic responses associated with the presence of a linker. Use of a PEG-aldehyde such as PEG-propionaldehyde or PEG-acetaldehyde results in the formation of an imine through any of the primary amines present on the protein. This imine is then reduced with sodium cyanoborohydride or sodium borohydride to convert the imine to a secondary amine.

It is preferable that the procedure use a molar excess of PEG-aldehyde, relative to the number of amines present on the protein. A preferred ratio is 0.08 to 24 and a more preferred ratio is 1. tb 10. The reactions are preferably performed between a pH 7.0 to 9.0 at 4° C. for 15 to 40 hours. However, lower pH values are recognized to restrict labeling to the N-terminus (pH. 5.0 to 7.0) and higher pH are recognized to enhance ε-amino group labeling on lysine residues (pH 9.0 to 10.0). Specific conditions required for PEGylation of non-glycosylated EPO and EPO analogs are set forth in Example 1.

PEGylation may be performed using PEG-aldehydes with the following formula: [R—O—(CH₂CH₂—O)_{X}—(CH₂)_{Y}—NH), wherein R is H or C₁ to C₄ alkyl, X is a number from about 70 to about 1200, preferably about 450 to about 1200, and even more preferrably about 450 to about 700, and Y is a number from 1 to 4.

The PEGylation reactions were run under conditions that permit the formation of an imine bond. Specifically, the pH of the solution ranged from 7 to 9 and methoxy-PEG-propionaldehyde concentrations ranged from 1 to 24 molar excess of the amine concentration. The PEGylation reactions were normally run at 4° C. to minimize degradation of the protein by other chemical and physical degradation processes. The different non-glycosylated EPO derivatives were isolated using size exclusion chromatography (SEC) (FIGS. 3 and 4).

Characterization of PEGylated Non-Glycosylated EPO Analogs

For illustrative purposes, the characterization of PEGylated MR-NGE-[W5K]-166Δand MR-NGE-166Δ with either PEG(5 kDa)-aldehyde or PEG (20 kDa)-aldehyde is discussed (Example 3). MR-NGE-[W5K]-166Δ and MR-NGE-166Δ contain 13 and 9 primary amines, respectively. Consequently, the PEGylation of these compounds with either the 5 kDa or 20 kDa methoxy-PEG-propionaldehyde polymer (PEG (5 kDa) aldehyde and PEG (20 kDa)-aldehyde, respectively) yield a variety of modified species that exhibit both in vivo and in vitro activity.

Species, PEGylated to varying degrees, were separated and enriched based on differences in hydrodynamic size. These pools were characterized using analytical HPLC-SEC, SDS-PAGE, and MALDI-Tof Mass Spectrometry. For example, the 5 kDa PEG modification of MR-NGE[W5K]-166Δ yielded three distinct pools with increasing degrees of PEGylation. Each distinct characterized pool was designated with a letter. The Compound B pool consisted of a mixture of primarily 5 kDa Tri-and Tetra-PEGylated species. The Compound C pool consisted of primarily 5 kDa Di-PEGylated species and the Compound D pool consisted of primarily 5 kDa Mono-PEGylated species. Compound E was unmodified MR-NGE[W5K]-166Δ.

The PEG (20 kDa) modification of MR-NGE[W5K]-166Δ yielded several-distinct pools with increasing degrees of PEGylation. The Compound F pool consisted of primarily 20 kDa PEGylated species (greater than Di-Pegylated) and the Compound G pool consisted of a blend of 20 kDa Mono-, Di-, and Tri-PEGylated species. In addition, the PEG (20 kDa) modification of MR-NGE-166Δ also yielded several distinct pools with increasing degrees of PEGylation. For example, Compound AH was isolated from a reaction mixture and consisted of primarily 20 kDa Tri-Pegylated species.

The degree of PEGylation and site of PEGylation can be controlled to primarily the N-terminus by lowering the pH and reducing the PEG:amine ratio. Reactions run at pH 7 and a 1.5:1.molar ratio of PEG-aldehyde:amine groups, preferentially react with the N-terminal α-amino group (Example 1g)., This is illustrated by a Lys_C enzymatic digestion of the Compound. Z pool which consists of primarily a 20 kDa Mono-PEGylated MR-NGE-166Δ species (Example 3). The Lys_C enzymatic digestion of Compound Z indicated that only the N-terminal peptide, liberated by the digestion, had a modified retention time.

Activity of PEGylated Non-Glycosylated EPO, Analogs:

These various PEGylated species were also tested for in vitro activity using a ³H-thymidine uptake assay in spleen cells (see Example 4). Mono-PEGylation with 5 kDa PEG generally has only a minor impact on in vitro activity; however, each-subsequent 5 kDa PEG addition (e.g. di-, tri-, and tetra-PEGylation) results in a.0.55 (±0.04) log reduction in in vitro activity (FIG. 9A). The impact of adding 20 kDa PEG moities.on in vitro activity is even greater, i.e., 0.63 (±0.09) log reduction in in vitro activity per 20 kDa PEG moiety attached (FIG. 9B).

Non-glycosylated PEGylated EPO analog derivatives were also tested for in vivo activity. In vivo results were monitored by assaying hematocrit levels in 5-8 week old B6C3F1 or CD-1 mice injected with various PEGylated species given at a single dose of either 10, 20, 50, or 100 μg/mouse/injection (FIGS. 10, 12, and 13; Tables V-VII; Example 5). The results clearly show, for dose 20 μg/kg, an increase in hematocrit relative to the PBS/BSA control. For comparison purposes, glycosylated EPO was also ran.

The results indicate that the PEGylated non-glycosylated samples have sufficient plasma half-lives to elicit a hematopoietic response unlike non-glycosylated EPO which showed no response due to rapid clearance from the plasma (FIG. 2). In addition, a comparison of in vivo activity at 7, 10, and 14 days following a single dose of a 20 kDa tri-PEGylated NGE (Compound AM), glycosylated EPO, or PBS show that only the tri-PEGylated NGE illicits a response beyond 7days indicating that this compound has an increased half-life relative to wild type glycosylated EPO (FIG. 13).

Furthermore, the degree of PEGylation and the size of the PEG moiety have an effect on in vivo activity. Generally, the greater the degree of PEGylation and/or the larger the size of the PEG entities the greater the in vivo activity (FIG. 10, Table V). Optimal in vivo activity was observed with PEG moieties>5 kDa in size (Table V) and proteins modified with an average of −3 PEG moieties (FIG. 12).

It is important to note that the in vitro and in vivo activities of the PEGylated proteins are inversely correlated (FIG. 11). The compounds with lower in vitro activity have higher in vivo activity. The data presented in support of the present invention suggests that the mere measurement of an in vitro activity is not necessarily predictive of performance in vivo.

Interestingly, estimates of bioavailability of Compounds C and G representing PEGylated pools of MR-NGE[W5K]-166Δ (discussed above) relative to non-glycosylated MR-NGE-1666 (FIG. 2) are 4.5% and 60%, respectively (FIG. 14, Table VIII). These results are surprisingly counter-intuitive, based on the hydrodynamic size of the two compounds. By SEC on TSK3000 column, Compound C has a main peak retention time that is similar to that observed with ˜121 kDa molecular weight protein and Compound G has a main peak retention time that is similar to that observed with ˜466 kDa molecular weight protein. Consequently, if the bioavailability is presumed to be primarily constrained by the hydrodynamic size of the protein, the bioavailability of C would have been expected to exceed that of Compound G. Thus, the bioavailability of these PEGylated compounds is being controlled by something other than hydrodynamic size. Clearly the results indicate that 20 kDa PEGylated species of non-glycosylated erythropoietin would be preferred over 5 kDa PEG modifications because of the extended plasma half-life and improved bioavailability (FIG. 14; Table VIII)

Thus, PEGylation can replace glycosylation as a means of extending plasma half-life by reducing the clearance rate of the non-glycosylated erythropoietin. Furthermore, the plasma half-life of the PEGylated species is greater than that which is achievable through natural glycosylation, due in part, to PEGylated species of EPO not being susceptible to the clearance mechanisms associated with the carbohydrate structures.

Solubility and Stability of Non-Glycosylated EPO Analog Derivatives:

The present invention provides not only modified proteins with erythropoietin activity that have a longer time-action but EPO analogs which have improved solubility and/or stability properties which may be especially useful for alternate delivery methodologies and formulation development. Thus, the pharmaceutical properties of the modified EPO analogs of the present invention make it possible to develop formulations that are more convenient and efficacious for the patient.

For example, the physical stability of Compound Z, which is characterized as consisting of primarily a 20 kDa mono-PEGylated MR-NGE-166Δ species was studied under stabilizing high ionic strength conditions (20 mM TRIS, 500 mM NaCl, 1 mM EDTA, pH 7.4), physiologic ionic strength conditions (20 mM TRIS, 150 mM NaCl, 1 mM EDTA, pH 7.4) , and “multi-use formulation” conditions (20 mM TRIS, 150 mM NaCl, 1 mM EDTA, 3.0 mg/mL m-cresol, pH 7.4). These studies indicated that the addition of a single 20 kDa PEG moiety at the N-terminus significantly improves the physical stability of non-glycosylated EPO (see Example 3, Table III)).

The present invention thus, provides non-glycosylated variants of erythropoietin modified to have improved biophysical properties. PEGylation can replace glycosylation as a means of extending plasma half-life by reducing the clearance rate of the non-glycosylated erythropoietin. This time extension is greater than that which is achievable through natural glycosylation, since the PEGylated species of proteins are not susceptible to the clearance mechanisms associated with the carbohydrate structures. Preferably, 20 kDa PEG-modified NGEAs are favored because of the greater extension of plasma half-life and, surprisingly, increased bioavailability. In addition, the derivatized analogs of the present invention provide improved solubility and/or stability making them useful in a manufacturing environment. Lastly, the discoveries associated with the present invention make it possible to develop formulations that are more convenient and efficacious for the patient. Moreover, the utilization of an E. coli derived source as opposed to mammalian cell-derived-source would reduce the cost of manufacturing and reduce the cost of the productto the consumer.

The following examples are presented to further describe the present invention. The scope of the present invention is not to be construed as merely consisting of the following examples. Those skilled in the art will recognize that the particlar reagents, equipment, and procedures described are merely illustrative and are not intended to limit the present invention in any manner.

### EXAMPLE 1

### PEGylation of EPO Analogs

PEG-propionaldehyde Modification of Non-Glycosylated EPO and EPO Analogs:

EPO analogs were reacted with 5 kDa and 20 kDa polyethylene glycol-aldehydes (PEG-aldehydes) to produce analogs covalently attached to ethylene groups. The PEGylated analogs were then separated into populations based on the extent of PEGylation and then various populations were tested to determine in vivo and in vitro activity. One skilled in the art would understand that the exemplified compounds can be made with or without an amino acid present at position 166.

1a: MR-NGE[W5K]-166Δ covalently attached to 5 kDa PEGS:

A 1.75 mL aliquot of a 2.36 mg/mL solution of MR-NGEIW5K]-166Δ was used to dissolve 35.70 mg of methoxy-PEG(5kDa)-aldehyde (Lot# PT-037-36, purchased from Shearwater Polymers, Inc. Huntsville, Ala.) (4.3:1 ratio of PEG:NH₂ groups). A 100 uL solution of 7.5 mg/mL NaCNBH₃ was added to reductively alkylate the Schiff's base that was generated upon the reaction of the aldehyde with the primary amine. The reaction was performed in 50 mM Borate, 150 mM NaCl, pH 9.0, at 4° C., for ˜40 hours. PEGylated species were then purified, characterized, and tested for activity (see Examples below).

1b: MR-NGE[W5K]-166Δ covalently attached to 20 kDa PEGs:

A 1.75 mL aliquot of a 2.36 mg/mL solution of MR-NGE[W5SK-1-6-6A was used to dissolve 134.6 mg of methoxy-PEG (20 kda)-aldehyde (Lot#PT-087-01, purchased from Shearwater Polymers, Inc., Huntsville, Ala.) (4.1:1 ratio of PEG:NH₂ groups). A 100 uL solution containing 7.5 mg/mL of NaCNBH₃ was added to reductively alkylate the Schiff's base that was generated upon the reaction of the aldehyde with the primary amine. The reaction was performed at in 50 mM Borate, 150 mM NaCl, pH 9.0, at 4° C.; for ˜40 hours. PEGylated species were then purified, characterized, and tested for activity (see Examples below).

1c: MR-NGE-166Δ covalently attached to 20 kDa PEGS:

A 3.7 mL aliquot of a 1.6.mg/mL solution of MR-NGE-166Δ was used to dissolve 86.3 mg of methoxy-PEG (20 kDa)-aldehyde (1.-5:1 ratio of PEG:NH₂ groups). A 36.2 uL solution containing 7.5 mg/mL of NaCNBH₃ was added to reductively alkylate the Schiff's base that was generated upon the reaction of the aldehyde with the primary amine. The reaction was performed at in Phosphate Buffered Saline, pH 7.0, at 4° C., for ˜40 hours. PEGylated products were purified and characterized as described below.

1d: MR-NGE-166Δ covalently attached to 5 kDa PEGS:

A 7.4 mL aliquot of a 0.94 mg/mL solution of MR-NGE-166Δ was used to dissolve 236.6 mg of methoxy-PEG (5 kDa)aldehyde (14:1 ratio of PEG:NH₂ groups). A 396.4 uL solution containing 7.5 mg/mL of NaCNBH₃ was added to reductively alkylate the Schiff's base that was generated upon the reaction of the aldehyde with the primary amine. The reaction was performed at in 50 mM Borate, Phosphate Buffered Saline, pH 9.0, at 4° C., for approximately 40 hours.

1e: MR-NGE-166Δ covalently attached to 20 kDa PEGs:

A 0.25 mL aliquot of a 0.5 mg/mL solution of MR-NGE-166Δ was used to dissolve 1.6 mg of methoxy-PEG (20 kDa)-aldehyde (1.3:1 ratio of PEG:NH₂ groups). A 0.7 uL aliquot of a 7.5 mg/mL solution of NaCNBH₃ was added to reductively alkylate the Schiff's base that was generated upon the reaction of the aldehyde with the primary amine. The reaction was performed at pH 9.0, at 4° C., for approximately 40 hours.

1f: MR-NGE-166Δ covalently attached to 20 kDa PEGs:

A 0.25 mL aliquot of a 1.54 mg/mL solution of MR-NGE-166Δ was used to dissolve 25.3 mg of methoxy-PEG (20 kDa)-aldehyde (6.8:1 ratio of PEG:NH₂ groups). A 10.6 uL aliquot of a 7.5 mg/mL solution of NaCNBH₃ was added to reductively alkylate the Schiff's base that was generated upon the reaction of the aldehyde with the primary amine. The reaction was performed at pH 9.0, at 4° C., for approximately 40 hours.

1g: MR-NGE-166Δ covalently attached to 20 kDa PEGs:

A 0.25 mL aliquot of a 1.72 mg/mL solution of MR-NGE-166Δ was used to dissolve 6.1 mg of methoxy-PEG (20 kDa)-aldehyde (1.5:1 ratio of PEG:NH₂ groups). A 2.6 uL aliquot of a 7.5 mg/mL solution of NaCNBH₃ was added to reductively alkylate the Schiff's base that was generated upon the reaction of the aldehyde with the primary amine. The reaction was performed at pH 7.0, at 4° C., for approximately 40 hours.

1h: MR-NGE-166Δ covalently attached to 20 kDa PEGS:

A 0.25 mL aliquot of a 0.5 mg/mL solution of MR-NGE-166Δ was used to dissolve 4.8 mg of methoxy-PEG (20 kDa)-aldehyde (4:1 ratio of PEG:NH₂ groups). A 2.0 uL aliquot of a 7.5 mg/mL solution of NaCNBH₃ was added to reductively alkylate the Schiff's base that was generated upon the reaction of the aldehyde with the primary amine. The reaction was performed at pH 7.0, at 4° C., for approximately 40 hours.

1j: MR-NGE-166Δ covalently attached to 20 kDa PEGs:

A 0.25 mL aliquot of a 1.33 mg/mL solution of Met-Arg-EPO was used to dissolve 9.2 mg of methoxy-PEG (20K)-aldehyde (3.6:1 ratio of PEG:NH₂ groups) A 3.9 uL aliquot of a 7.5 mg/mL solution of NaCNBH₃ was added to reductively alkylate the Schiff's base that was generated upon the reaction of the aldehyde with the primary amine. The reaction was performed at pH 8.0, at 4° C., for approximately 40 hours.

1k: MR-NGE-166Δ covalently attached to 20-kDa PEGs:

A 0.25 mL aliquot of a 1.33 mg/mL solution of MR-NGE-166Δ was used to dissolve 9.4 mg of methoxy-PEG (20 kDa)-aldehyde (2.9:1 ratio of PEG:NH₂ groups). A 3.9 uL aliquot of a 7.5 mg/mL solution of NaCNBH₃ was added to reductively alkylate the Schiff's base that was generated upon the reaction of the aldehyde with the primary amine. The reaction was performed at pH 8.0, at 4° C., for approximately 40 hours.

1l: MR-NGE-166Δ covlalently attached to 5 kDa PEGs:

A 1.8 mL aliquot of a 1.07 mg/mL solution of MR-NGE-166Δ was used to dissolve 4.7 mg of methoxy-PEG (5kDa)-aldehyde, 1:1 ratio of PEG:NH₂ groups, 7.8 uL of a 7.5 mg/mL solution of NaCNBH₃ was added to reductively alkylate the Schiff's base that was generated upon the reaction of the aldehyde with the primary amine. The reaction was performed in PBS at pH 7.0, 4° C. for ˜90 hours.

1m: MR-NGE[W5E]-166Δ covalently attached to 5 kDa PEGS:

A 1.8 mL aliquot of a 1.27 mg/mL solution of MR-NGECW5E]-166Δ was-used to dissolve 5.7 mg of methoxy-PEG (5 kDa)-aldehyde, 1:1 ratio of PEG:NH₂ groups, 9.3 uL of a 7.5 mg/mL solution of NaCNBH₃ was added to reductively alkylate the Schiff's base that was generated upon the reaction of the aldehyde with the primary amine. The reaction was performed in PBS at pH 7.0, 4° C., for ˜90 hours.

1n: MR-NGE[W5KK]-16E6A covalently attached to 5 kDa PEGs:

A 1.8 mL aliquot of a 0.82 mg/mL solution of MR-NGE[W5K]-166Δ was used to dissolve 3.7 mg of methoxy-PEG (5kDa)-7aldehyde, 0.7:1 ratio of PEG:NH₂ groups, 6.0 uL of a 7.5 mg/mL solution of NaCNBH₃ was added to reductively alkylate the Schiff's base that was generated upon the reaction of the aldehyde with the primary amine. The reaction was performed in PBS at pH 7.0, 4° C., for ˜90 hours.

1o: 4MR-NGE [W5E]-166Δ covalently attached to 20 kDa PEGs:

A 1.8 mL aliquot of a 1.27 mg/mL solution MR-NGE[W5E]-166Δ was use to dissolve 22.2 mg of methoxy-PEG (20 kDa)-aldehyde 1:1 ratio of PEG:NH₂groups; 9.3 uL of a 7.5 mg/mL solution of NaCNBH₃ was added to reductively alkylate the Shiff's base that was generated upon the reaction of the aldehyde with the primary amine. The reaction was performed in PBS at pH 7.0, 4° C., for ˜90 hours.

1p: MR-NGE[W5K]-166Δ covalently attached to 20 kDa PEGS:

A 1.8 mL aliquot of a 0.82 mg/mL solution of MR-NGE[W5K]-166Δ was used to dissolve 14.2 mg of methoxy-PEG(5kDa)-aldehyde, 0.6:1 ratio of PEG:NH₂ groups, 6.0 uL of a 7.5 mg/mL solution of NaCNBH₃ was added to reductively alkylate the Schiff's base that was generated upon the reaction of the aldehyde with the primary amine. The reaction was performed in PBS at pH 7.0, 4° C., for ˜90 hours.

1q: MR-NGE-166Δ covalently attached to 20 kDa PEGs:

A 2.8 mL aliquot of a 0.87 mg/mL solution of MR-NGE-166Δ was used to dissolve 36.7 mg of methoxyl-PEG(20 kDA)-SPA (N-hydroxysuccinimidyl ester of methoxypoly(ethylene glycol) propionic acid, MW 20000; Shearwater Polymers) in seven equal aliquots over 2.5 hours at 4C. This represents a 1.5:1 ratio of PEG:NH₂ groups. The reaction was performed in PBS at pH 7.0, 4C., for ˜19 hours. The material was purified by hydrophobic interaction chromatography on a TSK phenyl-5PW column (7.5 mm×7.5 cm) using a mobile phase consisting of 25 mM sodium phosphate and decreasing salt gradient of ammonium sulfate (loaded at 700 mM ammonium sulfate, pH 7.2). The collected material was further purified by size exclusion chromatography on a TSK 3000 column. The material eluting with the largest size was pooled and labeled as compound AH.

### EXAMPLE 2

### Purification and Folding of Non-Glycosylated EPO and EPO Analogs

Following induction with IPTG, 4 liter bacterial cultures containing host cells transformed with PET30_NGE constructs were spun down to obtain a bacterial pellet. This bacterial pellet was re-suspended in 180 mL of 50 mnM Tris/HCl pH 8.0. To the resuspended pellet, 80 mg of Lysozyme (Boehringer Mannheim, Lot number 84093121), 0.9 mL of 1M MgCl₂, 80 μL of DNAse I (1 mg/mL in 20 mM Tris, 50 mM NaCl, 50% glycerol, pH 7.4) were added. This mixture was stirred at RT for 30 minutes and then sonicated for 8 minutes with 5 second on pulse and 2 second of f pulse at settings of 100% tune and 6.5% amplitude in a Cole Parma Ultrasonic Homogenizer. The lysed material was spun down at 13,000 rpm in a Sorvall rotor for 20 minutes. The pellets were re-suspended in 120 mL of 0.1% Triton X-100, 5 mM EDTA using a PowerGen 700 Homogenizer from Fisher Scientific. The-material was spun down at 13,000 rpm in a Sorvall rotor for 20 minutes. The pellets were re-suspended in 60 mL of 0.1% Triton X-100, 5 mM EDTA and 60 mL of 0.5 M KC1 and then spun down at 13,000 rpm as stated before. The pellets were then washed with 120 mL of distilled H₂O and centrifuged at 13,000 rpm in a Sorvall rotor. These purified inclusion bodies were either used immediately or frozen at −20° C.

Inclusion bodies from 2 liters of induced bacterial culture were used as the starting material. These inclusion bodies were solubilized in 150 mL of 10 mM Tris, 5 mM cysteine, 7M urea, pH 7.0. Many mutants were soluble in these conditions. However, if a mutant remained cloudy under these conditions, the pH of the solublization mixture was adjusted to pH 9 or 10 for 30 min then readjusted the pH to between 7.0 and 7.4. This first step in the purification was achieved using a Waters 650E Advanced Protein Purification System. This material was then placed on either 180 mL column of Q Sepharose Fast Flow resin or SP Sepharose Fast Flow resin (Pharmacia) depending on the pI of 30 the protein which was being purified. The ion exchange column was equilibrated in 10 mM Tris, 5 mM cysteine, 7M urea, pH 7.0., The flow rate was 10 mL/min. The protein was eluted from the column using a linear gradient from 0 to 1 M NaCl over 55 minutes. The protein elution was monitored by UW absorbance at 280 nm. Fraction containing the protein of interest were pooled for refolding.

Refolding of non-glycosylated EPO analogs was successfully achieved by two different methods. The first method involved taking the pooled fractions from ion exchange and adjusting the protein concentration to 0.1 mg/mL. This material was then exhaustively dialyzed against 20 mM Tris, 0.5 M NaCl, pH 7.4 at 4° C. (approximately 1/10,000 dilution over 36 hrs). The other method (infinite dilution) involved taking the pooled fractions from the-ion exchange column and dripping the protein containing solution into a buffer containing 20 mM Tris, 500 mM NaCl, 35% glycerol, pH7.4 at ˜4° C. The drip rate was approximately 1 mL per 7 minutes and the final protein concentration in the refolding buffer was 0.1 mg/mL. The second methodology for refolding yields slightly more correctly folded protein. The refolded protein is then concentrated using a Millipore ProFlux M12 Tangential Flow Filtration System with a S3Y10 spiral cartridge. Once the material has been concentrated down to 300 mL to 400 mL, it is placed in a 2.5 L Amicon stir cell using YMl0 membrane and concentrated to between 40 mL and 80 mL.

The final step in the purification of NGEAs involves preparative size exclusion chromatography. The concentrated refolded protein (40 mL to 80 mL) was placed on a preparative Pharmacia Superdex 75 (60/600) column equilibrated with 20 mM Tris, L mM EDTA , 0.5 M NaCl, pH 7.4. The flow rate was 10 mL/min. The protein elution was monitored by UV absorbance at 280 nm. Fractions containing the purified protein were identified by SDS-PAGE, pooled, and stored at ˜80° C.

### EXAMPLE 3

### Purification and Characterization of PEGylated EPO Analogs

Preparative Size Exclusion Chromatography (SEC):

The purification of the various PEGylated species of non-glycosylated EPO and non-glycosylated EPO analogs was achieved by size exclusion chromatography on a Waters 650E Advanced-Protein Purification System: The protocol utilized a Superdex 200 (16/60) column equilibrated in 20 mM TRIS, 0.5M NaCl, lmM EDTA at pH 7.4. Typical, volume loads were less than or equal to to 2.5 mL. The flow rate was 1 mL/min and 1 mL fractions were collected. The column was run at room temperature. The protein elution was monitored by UV detector at 280 nm (FIGS. 3A and 4A).

Analytical Size Exclusion High Performance Liquid Chromatography (SEC-HPLC):

The PEGylation of non-glycosylated EPO analogs was monitored by SEC-HPLC, since the PEG modification makes a significant impact on the hydrodynamic radius of the protein. All analytical assays were performed on a HP1100 HPLC (Hewlett-Packard). The modification was monitored by either of two SEC-HPLC protocols.

The first protocol utilized a Superdex 200 (PC3.2/300) column equilibrated in 20 mM TRIS, 0.5 M NaCl, 1 mM EDTA at pH 7.4. The flow rate was 50 uL/min and 20 μg of protein was typically injected. The column was run at room temperature; however, the samples were maintained at 4° C. in the auto-injector. The protein elution was monitored by UV detector at 214 nm.

A second protocol utilized a TosoHaas G3000SWXL (7.8 mm ×30 cm) column equilibrated in 20 mM TRIS, 0.5M NaCl, 1 mM EDTA at pH 7.4 (FIG. 3B and 4B). The flow rate was 0.5 ML/min and 20 μg of protein was typically injected. The column was run at room temperature; however, the samples were maintained at 4° C. in the auto-injector. The protein elution was monitored by UV detector at 214 nm.

Sodium Dodecylsulfate-Polyacrylamide Gel Electrophorsis (SDS-PAGE):

SDS-PAGE was used to analyze preparative SEC fractions to facilitate pooling of species with similar degrees of PEGylation, as well as to characterize the final pooled products (FIG. 5). All SDS-PAGE analyses were performed on an Novex Powerease 500 system using Novex 16% Tris-Glycine. Pre-cast Gels were run using Novex Tris-Glycine SDS Running Buffer. The staining solution consisted of 0.05% Commassie Brilliant Blue (R250), 30% methanol, and 10% acetic acid. The de-staining solution was 30% methanol and 10% acetic acid. Novex Mark 12 Wide Range Protein Standards were routinely used as for molecular weight standards. Gels were run at 125 V until sample entered gel and then voltage was adjusted to 200V.

Matrix-Assisted Laser Desorption/Ionization-Time of Flight Mass Spectrometry (MALDI-TOF MS): All experiments were performed on a Micromass TofSpec-2E mass spectrometer fitted with Time Lag Focusing electronics, a Reflectron and Post Acceleration Detector (or P.A.D., used for high mass detection) The effective path length of the instrument in Linear mode is 1.2 meters, in Reflectron mode it is 2.3 meters.

Two dual micro-channel plate detectors are fitted for linear and reflectron mode detection. The laser used is a Laser Science Inc. VSL-337i nitrogen laser operating at 337 nm at 10 laser shots per second. All data were acquired using a 500 Mhz, 8 bit transient recorder and up to 100 laser shots were averaged per spectrum using the Post Acceleration Detector (when necessary to increase ion signal).

The detection efficiency of a micro-channel plate or electron multiplier reduces as the ion mass increases. The operation of these devices relies on the production of secondary electrons from the ion bombardment of a surface and this becomes less efficient as the ion impact velocity reduces. Higher mass ions have lower velocities than low mass ions with the same energy and hence produce less, or no, secondary ions. In order to enhance the detection of high mass ions previous studies have shown that the secondary ion species may be accelerated from a dynode surface place in the ion path into a conventional electron multiplier. The TofSpec-2-E has been modified such that an ion-to-ion conversion dynode:may be moved in and out of position in front of the standard micro-channel plate detector.

The net effect of the introduction of this ion-to-ion conversion dynode is a small increase in the single mass peak width and so the ability to move the dynode out of the ion path ensures that the resolution at low mass, where the detection efficiency is already high, need not be compromised.

Sinapinic acid was used as the ionization matrix as all masses observed were above 10 kDa. Mass appropriate reference proteins were used for internal and external calibration files in order to obtain accurate mass determinations, for the samples analyzed. Samples were all analyzed using a 1:2 sample to matrix dilution. Since the PEGylated samples were extremely heterogeneous they were always prepared in an attempt to obtain the highest concentration possible when spotted onto the plate.

The instrument was initially set up under the following linear high mass detector conditions for PEGylated samples:

| | |
|---|---|
| Source Voltage: 20.0 keV | Pulse Voltage: 3.6 kev |
| Extraction Voltage: 19.9 keV | Laser Course: 100 |
| Focus Voltage: 14.5 keV | Laser Fine: 60 |
| Linear detector: 3.5 keV | High mass detector: 12.0 keV |
| P.A.D.: (in line) | |

These settings were then modified (if needed) to give the best signal/noise ratio and highest resolution. Examples of MALDI-Tof MS analysis are shown in FIGS. 6 and 7.

Table II provides a characterization of different non-glycosylated EPO derivatives of MR-NGE[W5K]-166Δ and MR-NGE-166Δ modified with either PEG (5 kDa)-aldehyde or PEG (20 kDa)-aldehyde (Examples 1a, 1b, and 1g). Species in bold capitalized print were qualitatively identified as the major species using SDS-PAGE and MALDI-Tof analysis. Peak assignments by analytical HPLC-SEC were assigned based upon sequential correlation between degree of PEGylation and retention-time. The assignments were also correlated to the species observed by SDS-PAGE and MALDI-Tof analyses. The average degree of PEGylation is the percent-weighted average based upon the analytical HPLC-SEC. For species that were greater than or equal to a certain degree of modification the lowest integer value was used in the calculation.

TABLE II

| | | PEG | | | Analytical | Average |
|---|---|---|---|---|---|---|
| | | Size | SDS-PAGE | MALDI-Tof | HPLC-SEC | Degree of |
| Cmpd | Protein | (kDa)^{a} | Species | Species | Species | PEGylation |
| AH | MR-NGE- | 20-SPA | Di-, Tri-, | Di-, Tri-, | 100% Tri | 3.0 |
| | 166Δ | | Tetra- | Tetra- | | |
| AM | MR-NGE | 20 | Di-, Tri-, | Di-, Tri-, | 44.2% >Tetra- | 3.3 |
| | [W5K]- | | Tetra-, | Tetra- | 43.1% Tri- | |
| | 166Δ | | >Tetra | | 12.6% Di- | |
| B | MR-NGE | 5 | tri-, | mono-, di-, | 37.7% tetra- | 3.2 |
| | [W5K]- | | tetra-, | tri-, tetra-, | 30.7% tri- | |
| | 166Δ | | penta- | hexa-, | 11.6% ≧penta- | |
| | | | | septa-, | 11.0% di- | |
| | | | | octa- | | |
| C | MR-NGE | 5 | mono-, di-, | mono-, di-, | 55.1% di- | 2.4 |
| | [W5K]- | | tri- | tri-, penta-, | 18.9% tri- | |
| | 166Δ | | | hexa-, | 15.5% tetra- | |
| | | | | speta-, | 10.5% mono- | |
| | | | | octa- | | |
| D | MR-NGE | 5 | mono-, di-, | native, | 60.5% mono- | 1.5 |
| | [W5K]- | | native | mono-, di-, | 19.6% di- | |
| | 166Δ | | | penta-, | 16.0% tri- | |
| | | | | hexa-, | 3.8% unreacted | |
| | | | | septa- | | |
| F | MR-NGE | 20 | mono-, di-, | mono-, di-, | 64.6% ≧Tri- | 2.5 |
| | [W5K]- | | ≧tri- | tri- | 27.4% di- | |
| | 166Δ | | | | 5.0% mono- | |
| G | MR-NGE | 20 | mono-, di-, | mono-, di-, | 37.9% di- | 2.1 |
| | [W5K]- | | ≧tri- | tri- | 33.3% ≧tri- | |
| | 166Δ | | | | 28.8% mono- | |
| Z | MR-NGE- | 20 | mono- | mono-, | 85.2% mono- | 1.0 |
| | 166Δ | | | native | 7.3% di- | |
| | | | | | 1.4% ≧tri | |

^{a}All compounds with the exception of AH, were PEGylated using a propionaldehyde derivatized methoxy-PEG reagent. Compound AH was PEGylated using the succinimidyl derivative of methodxy-PEG propionic acid (see Example 1q).

Enzymatic Digest of PEGylated Species:

Endoproteinase Lys_C is a serine proteinase which specifically cleaves on the C-terminal side of lysine residues. A protein sample was typically digested at 200 μg/mL in a solution of 1M guanidine hydrochloride, 20 mM Tris, 1 mM EDTA, pH 8 containing 10 μg/mL Lys_C (Promega) for 3 hours at 37° C. The digested protein was reduced by addition of 10 mM DTT for 10 minutes at 37° C., then quenched by addition of 10% (v/v) of a 1% TFA solution. The resulting peptide fragments were separated by reversed-phase HPLC on a Zorbax-SB C8 column using a TFA/ACN mobile phase (A-buffer: 0.1% TFA, B-buffer: 0.1 TFA in 80% ACN) at 1 mL/min. The peptide mixture was injected onto the column equilibrated at 10% B, after five minutes the gradient was increase at 1%B per minute for 55 minutes. HPLC peaks were identified by MALDI-Tof, LC-MS, and/or N-terminal analysis.

As illustrated in FIG. 8, peptide mapping has been used to characterize N-terminally modified proteins. The peptide map of Compound Z reveals that peptide L1 is modified by the PEGylation process and elutes later and with a broader profile indicative of a peptide covalently modified with a heterogeneous, 20K PEG moiety. Note, the PEG(2.0 kDa)-L1 peptide has been identified as the N-terminal, fragment of MR-HIP, i.e., PEG (20K)-Met-Arg-Ala-Pro-Pro-Arg-Leu-Ile-Cys-Asp-Ser Ag-Val-Leu-Glu-Arg-Tyr-Leu-Leu-Glu-Ala-Lys.

Physical Stability Studies:

The physical stability of various proteins were studied using a Dynamic Light. Scattering (DLS) assay to monitor protein-aggregation as function of size. A protein solution was diluted to either a) 20 mM Tris, 500 mM NaCl, 1 mM EDTA (simulated processing conditions), b) 20 mM Tris, 150 mM-NaCl, 1 mM EDTA (simulated physiological conditions), or c) 20 mM Tris, 150 mM NaCl, 3 mg/mL m-Cresol, 1 mM EDTA (simulated formulation conditions), containing 0.1 mg/mL protein. Solution pH was adjusted to 7.4 (±0.05) with HCl/NaOH and filtered through a Millex-GV filter (4 mm, 0.2 g) into a 6×50 mm borasilica type-I glass tube. The average light-scatter intensity weighted particle size was collected on a Brookhaven BI900 Instrument consisting of a goniometer at a 90°, angle digital correlator, and a Lexel model 3500 argon ion laser adjusted to the 488-nm line. The experimentally determined autocorrelation function C(t) was analyzed by the cumulants method to yield the weight-averaged hydrodynamic diameter. A plot of hydrodynamic diameter versus incubation time at 37° C. resulted in an exponential-like curve. The time before a significant change in particle size, or lag time, was determined by fitting linear lines to the pre-growth and growth phase data points. The intersection was defined as the lag time.

The physical stability of MR-NGE-166Δ and N-terminal 20 kDa mono-PEGylated MR-NGE-166Δ was monitored by dynamic light scattering under high ionic strength, physiological ionic strength, and formulation conditions. The pH and temperature of the experiments were 7.4 and 37° C., respectively. The results are presented in Table III.

Aggregation was assessed by changes in size determined by quadratic fits to the auto-correlation function. The data in Table III show that N-terminal PEGylation of MR-NGE-166Δwith 20 kDa moiety dramatically increases the lag time before aggregation begins and slows the growth rate of aggregation after it has initiated.

TABLE III

| | | | N-terminal 20 kDa | |
|---|---|---|---|---|
| | | | mono-PEGylated MR- | |
| | MR-NGE-166Δ | | NGE-166Δ | |
| | | Growth | | Growth |
| Solvent | Lag Time | Rate | Lag Time | Rate |
| Condition | (Hr) | (nm/Hr) | (Hr) | (nm/Hr) |
| 500 mM NaCl | 9.5 | 124 | >74 | — |
| 150 mM NaCl | 0.1 | 334 | 83 | 0.07 |
| 150 mM NaCl + m- | * | * | 28 | 41 |
| Cresol | | | | |

* Solution immediately precipitated upon cresol addition

### EXAMPLE 4

### In Vitro Activity of Non-Glycosylated EPO Analogs With and Without PEGylation

In vitro Assay:

The bioactivity was determined using the method of Krystal [17]. Briefly, B6CF1 mice were treated with daily injections of phenylhydrazine (60 mg/kg) for two consecutive days. Phenylhydrazine treatment induce a 10× increase in spleen size and renders an organ enriched with EPO responsive cells. On the third day, the spleens are removed and teased into Alpha MEM without ribonucleosides and deoxyribonucleosides. The cell suspension is then filtered through a 200-gauge nylon mesh, cell density determined, and a spleen cell mixture prepared. The spleen cell mixture contained 4×10⁶ cells/mL spleen cells, 20% fetal calf serum (FCS), and 0.1 mM b-mercaptoethanol (BME) in a-MEM without ribonucleosides and deoxyribonucleosides (a-N). The spleen cell mixture was plated into microtiter plates at volume of 0.05 mL/well. The NEG, NEGA, PEGylated NGE, 4 PEGylated NEGA and EPOGEN® test samples were added to each well.

Each test sample was diluted with bioassay medium containing 78% Alpha-MEM, 20% heat-inactivated FCS, 1% BME and 1% penicillin/streptomycin/fungizone and 0.05 mL was added per well. Sample dilutions ranged from 10¹ to 10⁹. Cultures were incubated for 22 hrs at 37° C. in a humidified atmosphere of 5% CO2; 95% air. After this initial incubation, 20uL of ³H-thymidine stock containing ˜50uCi/mL in a-N was added to each well. The culture were then incubated at 37° C. for an additional two hours. The cellular contents were then harvested by filtration using a glass fiber filter impregnated with scintillant. The cellular rentate was washed with distilled water and, the filters dried-with methanol. The extent of ³H-thymidine into DNA stimulated by NEG, NEGA, PEGylated NGE, PEGylated NEGA and EPOGEN® test samples was determined on a Beckman LS3801 liquid scintillation counter. The standard curve was calibrated against the World Health Organization Second International Reference Preparation.

PEGylation of MR-NGE[W5K]-166Δ and MR-NGE-166Δ yielded a variety of modified species that exhibit both in vivo and in vitro activity. Non-glycosylated EPO analogs were modified as described in Example 1 and separated into various species pools based on the degree of PEGylation. Size Exclusion Chromatography as described in Example 3 was used to separate the PEGylated species. The in vitro results obtained for the ³H-thymidine assay described above are presented in Table IV and FIG. 9.

TABLE IV
In vitro activity, as monitored by the ³H-
thymidine uptake assay in spleen cells, of various
compounds.

| | | Approx. | PEG | | | Std |
|---|---|---|---|---|---|---|
| | | # of | Size | log | Std | Err |
| Compound | Protein | PEG | (kDa)^{a} | (U/mg) | Dev | Mean |
| MRNGE166Δ | std | 0 | | 5.12 | 0.13 | 0.05 |
| MRNGE166Δ | std | 0 | | 5.35 | 0.25 | 0.08 |
| MRNGE166Δ | std | 0 | | 5.34 | 0.06 | 0.04 |
| MRNGE166Δ | std | 0 | | 5.73 | 0.17 | 0.05 |
| MRNGE166Δ | std | 0 | | 5.37 | 0.11 | 0.03 |
| AA | MRNGE | 0 | 20 | 6.12 | 0.18 | 0.04 |
| | 166Δ | | | | | |
| AB | MRNGE | 1.0 | 5 | 5.50 | 0.34 | 0.09 |
| | 166Δ | | | | | |
| AC | MRNGE | 1.0 | 5 | 5.59 | 0.18 | 0.05 |
| | [W5E] | | | | | |
| | 166Δ | | | | | |
| AD | MRNGE | 1.0 | 5 | 5.75 | 0.28 | 0.08 |
| | [W5K] | | | | | |
| | 166Δ | | | | | |
| AE | MRNGE | 1.0 | 20 | 5.38 | 0.22 | 0.06 |
| | [W5E] | | | | | |
| | 166Δ | | | | | |
| AF | MRNGE | 1.0 | 20 | 5.48 | 0.41 | 0.15 |
| | [W5K] | | | | | |
| | 166Δ | | | | | |
| AH | MRNGE | 3.0 | 20-SPA^{a} | 2.19 | 0.17 | 0.06 |
| | 166Δ | | | | | |
| AI | MRNGE | 2.4 | 20-SPA | 3.23 | 0.13 | 0.04 |
| | 166Δ | | | | | |
| AJ | MRNGE | 1.6 | 20-SPA | 4.56 | 0.29 | 0.08 |
| | 166Δ | | | | | |
| AK | MRNGE | 2.5 | 20 | 3.18 | 0.76 | 0.18 |
| | [W5K] | | | | | |
| | 166Δ | | | | | |
| AL | MRNGE | 2.8 | 20 | 3.30 | 0.35 | 0.10 |
| | [W5K] | | | | | |
| | 166Δ | | | | | |
| AM | MRNGE | 3.3 | 20 | 3.31 | 0.34 | 0.11 |
| | [W5K] | | | | | |
| | 166Δ | | | | | |
| B | MRNGE | 3.2 | 5 | 4.26 | 0.13 | 0.03 |
| | [W5K] | | | | | |
| | 166Δ | | | | | |
| C | MRNGE | 2.4 | 5 | 5.09 | 0.09 | 0.03 |
| | [W5K] | | | | | |
| | 166Δ | | | | | |
| D | MRNGE | 1.5 | 5 | 5.16 | 0.07 | 0.02 |
| | [W5K] | | | | | |
| | 166Δ | | | | | |
| F | MRNGE | 2.5 | 20 | 3.59 | 0.09 | 0.03 |
| | [W5K] | | | | | |
| | 166Δ | | | | | |
| H | MRNGE | 8.0 | 5 | 1.26 | 0.02 | 0.01 |
| | [W5K] | | | | | |
| | 166Δ | | | | | |
| I | MRNGE | 4.5 | 5 | 2.80 | 0.07 | 0.03 |
| | [W5K] | | | | | |
| | 166Δ | | | | | |
| J | MRNGE | 3.0 | 5 | 3.86 | 0.07 | 0.03 |
| | [W5K] | | | | | |
| | 166Δ | | | | | |
| K | MRNGE | 2.0 | 5 | 4.65 | 0.03 | 0.02 |
| | [W5K] | | | | | |
| | 166Δ | | | | | |
| L | MRNGE | 1.0 | 5 | 5.18 | 0.05 | 0.03 |
| | [W5K] | | | | | |
| | 166Δ | | | | | |
| M | MRNGE | 0.0 | 5 | 5.06 | 0.06 | 0.04 |
| | [W5K] | | | | | |
| | 166Δ | | | | | |
| N | MRNGE | 7.0 | 5 | 1.00 | | |
| | 166Δ | | | | | |
| O | MRNGE | 4.5 | 5 | 2.78 | 0.11 | 0.03 |
| | 166Δ | | | | | |
| P | MRNGE | 2.5 | 5 | 3.77 | 0.11 | 0.05 |
| | 166Δ | | | | | |
| Q | MRNGE | 2.0 | 5 | 4.04 | 0.09 | 0.05 |
| | 166Δ | | | | | |
| R | MRNGE | 1.0 | 5 | 5.12 | 0.13 | 0.04 |
| | 166Δ | | | | | |
| S | MRNGE | 0.0 | 5 | 5.23 | 0.12 | 0.04 |
| | 166Δ | | | | | |
| T | MRNGE | 3.8 | 5 | 3.13 | 0.38 | 0.17 |
| | 166Δ | | | | | |
| U | MRNGE | 3.3 | 5 | 3.65 | 0.28 | 0.12 |
| | 166Δ | | | | | |
| V | MRNGE | 2.3 | 5 | 3.89 | 0.28 | 0.10 |
| | 166Δ | | | | | |
| W | MRNGE | 1.8 | 5 | 4.90 | 0.11 | 0.03 |
| | 166Δ | | | | | |
| X | MRNGE | 1.2 | 5 | 5.07 | 0.22 | 0.06 |
| | 166Δ | | | | | |
| Y | MRNGE | 1.9 | 20 | 4.42 | 0.12 | 0.03 |
| | 166Δ | | | | | |
| Z | MRNGE | 1.0 | 20 | 4.90 | 0.15 | 0.04 |
| | 166Δ | | | | | |

^{a}All compounds with the exception of AH, AI, and AJ, were PEGylated using a propionaldeyde derviativzed methoxy-PEG reagent. Compounds AH, AI, and AJ were PEGylated using the succinimidyl derivative of methoxy-PEG propionic acid.

### EXAMPLE 5

### In Vivo Testing of Non-Glycosylated EPO Analogs with and Without PEGylation:

In Vivo Assay:

Groups of four to six female B6CF1,C57BL/6J, or CD-1 mice, ranging in age from 5-8 weeks, were used to assess in viavoactivity. Changes in hematocrit levels were used as the indicator of in vivo activity. To establish a baseline prior to dosing, the mice were anesthetized and the baseline hemoatocrit established by filling two heperinized micro-hematocrit tubes from the retro-prbital venous plexus. Each animal was weighted and marked for identification through the study. The mice received either a single subcutaneous injection per week of PEGylated NGE, PEGylated NEGA, or EPOGEN®. PEGylated NGE, PEGylated NEGA, and EPOGENs were dosed at either 10, 20, 50, or 100 ug/kg. Hematocrit levels were determined on days 0, 7, 10, and 14. Control animals were injected with PBS containing 2.5% BSA. PEGlation of MR-NGE[W5K]-166® and MR-NGE-166® yielded a variety of modified species that exhibited both in vivo and in vitro activity. Non-glycosylated EPO analogs were modified as described in Example 1 and separated into various species pools based on the degree of PEGylation. Size Exclusion Chromatography,as described in Example 3 was used to separate the PEGylated species. The in vivo results

TABLE V
Change in hematocrit after a single, s.c. -
administered dose of compound on day 0. Hematocrit
measurements were performed on days 0 and 7. The difference
between day 7 and day 0 is reported as Delta within this
table.

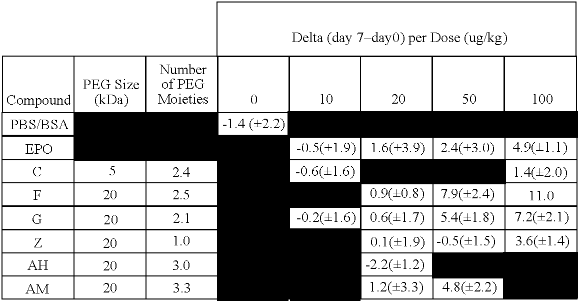

were monitored by hematocrit levels as described above and are presented in Tables V, VI, and VII.

TABLE VI
Change in hematocrit after a single, subcutaneous
administered dose of the compound at Day 0. Hematocrit
measurements were performed on days 0, 7, and 10. The
difference between day 10 and day 0 is reported as Delta
within this table.

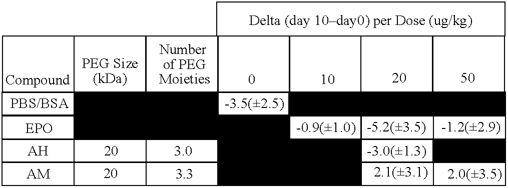

TABLE VII
Change in hematocrit after a single, s.c. -
administered dose of the compound at Day 0. Hematocrit
measurements were performed on days 0, 7, 10, and 14. The
difference between day 14 and day 0 is reported as Delta
within this table.

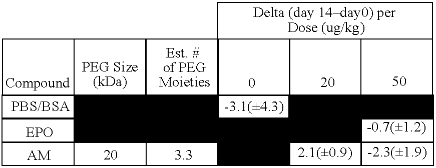

Pharmacokinetic Assay: Compounds C and G were administered by either intravenous or subcutaneous injection. Fischer 334 male rats were dosed at 10 Vg/kg levels. Three animals were used per time point per compound. Blood samples were collected at 15 min, 30 min, 1, 3, 5, 8,-24, and 48 hours after dosing for intravenous dosing. Blood samples were collected at 0, 2, 5, 8, 24, 48, 72, 96, and 120 hours after subcutaneous dosing.

A sandwich ELISA assay was used to measure concentrations of erythropoietin-like immunoreactivity in the plasma. The assays were validated to the following levels:

Compound C: Upper limit of quantitiation 600 pg/mL.

Lower limit of quantitiation 25 pg/mL Compound G: Upper limit of quantitation 1200 pg/mL.

Lower limit of quantitation 100 pg/mL

The pharmacokinetic results are presented in Table VIII and FIG. 14. A similar protocol was used to collect the data present in FIG. 2.

TABLE VIII
Pharmacokinetic Parameters of Erythropoietin-
like Immunoreactivity in Male Fischer 344 Rats After IV or
SQ Administration of Compounds C and G (Table II).

| | Compound C | | Compound G | |
|---|---|---|---|---|
| Statistics\Subject | IV | SQ | IV | SQ |
| AUC (pg * hr/mL)& | 38247 | 17943 | 751771 | 186546 |
| AUC (pg * hr/mL)% | — | 18182 | — | 239999 |
| HalfLife (h) | 10 | 11 | NC* | NC* |
| C_{Max} (pg/mL) | 86931 | 576 | 126132 | 3904 |
| T_{Max} (h) | 0.25 | 5 | 0.25 | 48 |
| Absolute | — | 47 | — | 25 |
| Bioavailability\hat{ } | | | | |
| Relative | — | 4.4 | — | 46 |
| Bioavailability# | | | | |

&AUC from 0-48 h
%AUC from 0-120 h
*No calculation, insufficient terminal data points
\hat{ }AUC SC₍₀₋₄₈ₕ₎/AUC IV₍₀₋₄₈ₕ₎
#(AUC G SC₀₋₄₈ₕ)/(AUC EPOGEN SC₀₋₄₈ₕ)

1. A protein selected from the group consisting of:
a) NGE;
b) NGE[5E];
c) MR-NGE;
d) MR-NGE-88E;
e) MR-NGE-88K;
f) MR-NGE-88P;
g) MR-NGE-88S;
h) MR-NGE[4E];
i) MR-NGE[5E];
j) MR-NGE[5K];
k) MR-NGE[W5E]; and
l) MR-NGE[W5K].

2. A protein selected from the group consisting of:
a) NGE-166Δ;
b) NGE[5E]-166Δ;
c) MR-NGE-166Δ;
d) MR-NGE-88E-166Δ;
e) MR-NGE-88K-166Δ;
f) MR-NGE-88P-166Δ;
g) MR-NGE-88S-166Δ;
h) MR-NGE[4E]-166Δ;
i) MR-NGE[5E]-166Δ;
j) MR-NGE[5K]-166Δ;
k) MR-NGE[W5E]-166Δ; and
I) MR-NGE[W5K]-166Δ.

3. The protein of claim 2, wherein the protein is MR-NGE-166Δ.

4. (canceled)

5. (canceled)

6. An erythropoietic compound having a protein portion and a polymer portion, wherein the protein portion is selected from the group consisting of: non-glycosylated human erythropoietin and non-glycosylated erythropoietin analogs and wherein the polymer portion consists of 1 to 5 polymer chains of the formula:
[R—O—(CH₂CH₂—O)ₓ—(CH₂)_{y}−NH]
wherein R is H or C₁ to C₄ alkyl, X is a number from about 70 to about 1200, and Y is a number from 1 to 4; and the polymer chain is covalently bonded to the protein portion by a secondary amine bond.

7. The erythropoietic compound of claim 6 wherein X is a number from about 225 to about 1200.

8. The erythropoietic compound of claim 7 wherein X is a number from about 340 to about 1200.

9. The erythropoietic compound of claim 8 wherein X is a number from about 450 to about 1200.

10. The erythropoietic compound of claim 9 wherein X is a number from about 450 to about 700.

11. The erythropoietic compound of claims 6 through 10 wherein the protein portion is a non-glycosylated erythropoietin analog and the polymer portion is bound to the protein portion at the N-terminus of the protein.

12. The erythropoietic compound of claim 6 through 10 wherein the protein portion is a protein of claim 1.

13. The erythropoietic compound of claim 6 through 10 wherein the protein portion is a protein of claim 2.

14. canceled

15. The erythropoietic compound of claim 6 through 10 made by a process comprising the steps of:
a) adding a polyethylene glycol-aldehyde polymer to a solution of non-glycosylated erythropoietic protein under conditions that permit the formation of an imine bond between an amino group of the protein and the aldehyde group of the polymer; and
b) adding a reducing agent to reduce the imine bond to a secondary amine bond.

16-28. (canceled)

29. A method for increasing the hematocrit levels in a mammal comprising the administration of a therapeutically effective amount of an erythropoietic compound of claims 6 through 10.

30. canceled

31. A pharmaceutical formulation adapted for the treatment of patients with insufficient hematocrit levels comprising an erythropoietic compound of claim 6.
